(19) United States
(12) Patent Application Publication
(10) Pub. No.: US 2017/0290886 A1
(43) Pub. Date: Oct. 12, 2017

(54) TREATMENT OF NEURODEVELOPMENTAL DISORDERS

(71) Applicant: Academia Sinica, Taipei (TW)

(72) Inventors: Eminy H.Y. LEE, Taipei (TW); Derek Jui-Cheng TAI, Taipei (TW); Yen-Chen LIU, Taipei (TW); Wei-Lun HSU, Taipei (TW)

(21) Appl. No.: 15/480,245

(22) Filed: Apr. 5, 2017

Related U.S. Application Data
(60) Provisional application No. 62/318,769, filed on Apr. 6, 2016.

(51) Int. Cl. A61K 38/17; A61K 31/191; A61K 38/18

(52) U.S. Cl. CPC A61K 38/1709; A61K 38/18; A61K 31/191

(57) ABSTRACT

Disclosed herein are methods for treating a neurodevelopmental disorder in a subject in need thereof. The method includes administering to the subject in need thereof an effective amount of a methyl-CpG-binding protein 2 (MeCP2) or a nucleic acid encoding the MeCP2 to alleviate or ameliorate the symptoms associated with the neurodevelopmental disorder. According to preferred embodiments, the MeCP2 has one or more post-translational modifications that result in increased levels of sumoylation, phosphorylation or both, as compared with that of the endogenous MeCP2 in the subject.

### CROSS-REFERENCES TO RELATED APPLICATIONS

The present application claims priority under 35 USC section 119(e) to U.S. Provisional Application No. 62/318,769, filed Apr. 6, 2016, which is incorporated by reference herein as if fully set forth.

### BACKGROUND OF THE INVENTION

### 1. Field of the Invention

The present disclosure in general relates to treatment of a neurodevelopmental disorder, e.g., Rett (RTT) syndrome.

### 2. Description of Related Art

The methyl-CpG-binding protein 2 (MeCP2) gene, MECP2, is an X-linked gene encoding the MeCP2 protein that contains 486 amino acids with two major structurally conserved domains, the methyl-DNA binding domain (MBD) (85 amino acids, a.a. 78-162) and the transcriptional repression domain (TRD) (104 amino acids, a.a. 207-310). MeCP2 functions as a transcriptional repressor by binding to the CpG island of methylated DNA and by recruiting co-repressors, such as histone deacetylase 1 (HDAC1) and Sin3a. Previous study indicated that MeCP2 is a multi-functional chromatin-associated protein that regulates the expression of various genes and it could function as either a transcriptional repressor or activator.

MeCP2 plays an important role in several neuro-developmental disorders, such as Rett syndrome (RTT), which is an autism spectrum disorder (ASD) caused by mutations of the MECP2 gene and 218 mutations have been identified that are linked to RTT. Patients with RTT usually develop normally before 18 months of age, but abnormal behaviors and regression develop afterwards that often include motor and language deficits, cognitive impairment, mental retardation and autism-like behaviors. Similar behavioral impairments were seen in mice with truncated MeCP2 and in mouse model of RTT with MeCP2 mutations at T158 and R306. Moreover, learning and memory function as well as synaptic plasticity were found impaired in a truncated MeCP2 mouse model of RTT.

In view of the above, there exists in the related art a need of a means that could restore MeCP2 level or its function in RTT patient, thus may serve as a potential candidate for the development of a medicament for treating neurodevelopmental diseases, disorders, and/or conditions, including RTT.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In general, the present disclosure relates to the unexpected discovery that the expression of a wild type methyl-CpG-binding protein 2 (MeCP2) or a modified MeCP2 in a MeCP2 knockout mouse model successfully rescues the deficits of social interaction, fear memory, and long-term potentiation (LTP), thereby improves social interaction, memory performance and synaptic plasticity in the MeCP2 knockout mouse. Thus, the wild type MeCP2 or the modified MeCP2 may serve as a candidate drug for developing medicaments suitable for treating neurodevelopmental disorders.

It is therefore the first aspect of the present disclosure to provide a method for treating a subject suffering from a neurodevelopmental disorder. The method includes the step of, administering to the subject an effective amount of MeCP2 or a nucleic acid encoding the MeCP2 to alleviate or ameliorate the symptoms associated with the neurodevelopmental disorder.

According to certain embodiments, the MeCP2 has one or more post-translational modifications that result in increased level of sumoylation, phosphorylation or both, as compared with that of the endogenous MeCP2 in the subject. According to one embodiment, the post-translational modification corresponds to sumoylation of the amino acid at position 412 in the wild-type MeCP2. According to another embodiment, the post-translational modification corresponds to phosphorylation of the amino acid at positions 308 or 421 in the wild-type MeCP2.

According to certain embodiments, the MeCP2 is administered to the subject in the amount of 0.001-100 mg/Kg. Preferably, the MeCP2 is administered to the subject in the amount of 0.01-80 mg/Kg.

According to some embodiments, the nucleic acid encoding the MeCP2 is an expression vector. The expression vector may be derived from a virus selected from the group consisting of, a herpes virus, a retrovirus, a vaccinia virus, an attenuated vaccinia virus, a canary pox virus, an adenovirus, and an adeno-associated virus. According to one embodiment, the expression vector, when being transducted into a subject, successfully encodes the MeCP2, in which the amino acid residue at position 412 is sumoylated. According to another embodiment, the expression vector, when being transducted into a subject, successfully encodes the MeCP2, in which the amino acid residue at positions 308 or 421 is phosphorylated. According to further embodiment, the expression vector, when being transducted into a subject, successfully encodes the MeCP2, in which the amino acid residue at positions 308 or 421 is phosphorylated; and the amino acid residue at position 412 is sumoylated.

According to certain embodiments, the neurodevelopmental disorder is any of attention deficit hyperactivity disorder (ADHD), schizophrenia, obsessive-compulsive disorder (OCD), mental retardation, autistic spectrum disorders, cerebral palsy, articulation disorder, Rett syndrome, or learning disabilities. In one preferred embodiment, the neurodevelopmental disorder is Rett syndrome.

According to optional embodiments, the method further includes the step of administering an effective amount of N-methyl-D-aspartate (NMDA), an insulin-like growth factor (IGF-1), or corticotropin-releasing factor (CRF) to the subject.

Accordance to embodiments of the present disclosure, the subject is human.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIGS. 1(a)-(1d) Identification of candidate SUMO sites on MeCP2. (a) In vitro SUMOylation assay showing MeCP2 SUMOylation by PIAS1. Purified GST-E1, His-E2, His-PIAS1, GST-MeCP2 and GST-SENP1 proteins were added to the reaction for this assay. (b) V5-MeCP2 plasmid and Myc-SUMO1 plasmid were co-transfected with different amount of Flag-PIAS1 plasmid to HEK293T cells with confirming MeCP2 SUMOylation by PIAS1. (c) Flag-PIAS1 and Myc-SUMO1 (or Myc-SUMO1δGG) plasmids were co-transfected with V5-MeCP2WT or different V5-MeCP2 lysine mutant plasmids to HEK293T cells. MeCP2 SUMOylation was examined by immunoblotting using anti-V5 antibody. The quantified result is shown in the lower panel (n=2 each group; F_{9,10}=240.59, #P<0.001; q=12.73, #P<0.001 comparing lane 3 vs. lane 9; q=21.1, #P<0.001 comparing lane 3 vs. lane 10; q=8.37, #P <0.01 comparing lane 9 vs. lane 10, one-way ANOVA followed by Newman-Keul posthoc multiple comparisons). (d) V5-MeCP2 or V5-MeCP2K412R plasmid was co-transfected with Myc-SUMO1 and Flag-PIAS1 plasmids to HEK293T cells. The cell lysate was immunoprecipitated with anit-Myc antibody and immunoblotted with anti-V5 antibody for confirmation of MeCP2 SUMOylation at Lys-412. The arrow indicates MeCP2 SUMOylation at Lys-412. All experiments are in two repeats. Data are expressed as mean±SEM.

FIGS. 2(a)-2(h) MeCP2 is associated with PIAS1 and can be sumoylated by PIAS1 at Lys-412 in rat hippocampus. (a) Co-IP experiment showing PIAS1 association with MeCP2 and vice versa in rat CA1 area. Experiments are in two repeats. (b) Immunohistochemistry showing PIAS1 and MeCP2 are both present in the nucleus of the same neurons in CA1 area of the rat brain. N=3. Scale bar equals 25 μm for the upper panel and scale bar equals 10 μm for the lower panel. (c) Immunocytochemistry showing PIAS1 and MeCP2 are co-localized in the nucleus of the same neuron from rat hippocampal cluture. Scale bar equals 5 μm. Experiments are in two repeats. (d) Flag-vector, Flag-MeCP2WT (with or without the addition of SUMO1 mutant protein) and Flag-MeCP2K412R plasmids were transfected to rat CA1 area and in vitro SUMOylation assay was carried out 48 h later to determine MeCP2 SUMOylation at Lys-412 in the hippocampus. Left panel: immunoblotted with anti-MeCP2 antibody. Right panel: immunoblotted with anti-SUMO1 antibody. Plasmid transfection and expression was confirmed by western blot using anti-Flag antibody. The quantified result is shown in (f) (n=4 each group; F_{3,12}=76.75, #P <0.001; q=16.5, #P<0.001 comparing the Flag-MeCP2WT group with Flag-vector group; q=18.39, #P<0.001 comparing the Flag-MeCP2K412R group with Flag-MeCP2WT group; q=17.47, ^{#}P <0.001 comparing the Flag-MeCP2WT+SUMO1 mutant group with Flag-MeCP2WT group, one-way ANOVA followed by post-hoc Newman-Keul multiple comparisons). (e) Cell lysates from the same plasmid transfections as described in (d) were immunoprecipitated with anti-MeCP2 antibody and immunoblotted with anti-ubiquitin antibody. Ub-MeCP2: ubiquitinated MeCP2. (g) Control siRNA or PIAS1 siRNA (8 pmol) was transfected to the CA1 area in rat brain and endogenous MeCP2 SUMOylation was determined by in vitro SUMOylation assay. (h) The quantified result of MeCP2 SUMOylation is shown in the upper panel (n=6 each group; t_{1,10}=9.65, #P<0.001, Student's t-test). The level of PIAS1 expression after PIAS1 siRNA transfection was determined by western blot, and the quantified result is shown in the lower panel (n=6 each group; t_{1,10}=11.38, ^{#}P<0.001, Student's t-test). Data are expressed as mean±SEM.

FIGS. 3(a)-3(d) MeCP2 phosphorylation facilitates MeCP2 SUMOylation and MeCP2 SUMOylation is induced by NMDA and IGF-1 and CRF treatments in the hippocampus. (a) Flag-vector, Flag-MeCP2WT, Flag-MeCP2S421A or Flag-MeCP2T308A plasmid was transfected to rat CA1 area and MeCP2 SUMOylation was determined by in vitro SUMOylation assay and quantified (n=4 each group; F_{3,12}=40.85, #P<0.001; q=12.02, #P<0.001, Flag-MeCP2WT group vs Flag-vector group; q=12.1, #P<0.001, Flag-MeCP2S421A group vs Flag-MeCP2WT group; q=13.88, #P<0.001, Flag-MeCP2T308A group vs Flag-MeCP2WT group). (b) The same plasmids were transfected to rat CA1 area as described above. NMDA (2 μg/μl) was administered 47 h after plasmid transfection. An additional group with Flag-vector transfection+PBS injection served as the control group. MeCP2 SUMOylation was determined by in vitro SUMOylation assay and MeCP2 phosphorylation at Ser-421 was determined by western blot using anti-phospho-Ser421MeCP2 antibody 1 h after NMDA injection (For MeCP2 SUMOylation, n=4 each group; F_{4,15}=74.65, #P<0.001; q=12.7, #P <0.001, Flag-vector+NMDA group vs Flag-vector+PBS group; q=4.65, **P<0.01, Flag-MeCP2WT+NMDA group vs Flag-vector+NMDA group; q=12.15, #P<0.001, Flag-MeCP2S421A+NMDA group vs Flag-vector+NMDA group; q=14.42, #P<0.001, Flag-MeCP2T308A+NMDA group vs Flag-vector+NMDA group; For MeCP2 phosphorylation at Ser−421, n=4 each group; F_{4,15}=23.65, #P <0.001; q=5.97, **P<0.01 comparing the Flag-vector+NMDA group with Flag-vector+PBS group; q=4.59, **P<0.01, Flag-MeCP2WT+NMDA group vs Flag-vector+NMDA group; q=8.17, #P<0.001, Flag-MeCP2S421A+NMDA group vs Flag-MeCP2WT+NMDA group; q=3.58, *P<0.05, Flag-MeCP2S421A+NMDA group vs Flag-vector+NMDA group). (c) PBS or IGF-1 (100 ng/ml) was injected to rat CA1 area and MeCP2 SUMOylation was determined 1 h later. Left panel: immunoblotted with anti-MeCP2 antibody. Right panel: immunoblotted with anti-SUMO1 antibody. (n=6 each group; t_{1,10}=15.9, #P<0.001). (d) PBS or CRF (100 ng/pl) was injected to rat CA1 area and MeCP2 SUMOylation was determined 1 h later (n=6 each group; t_{1,10}=10.91, #P <0.001). (e) DMSO or dexamethasone (30 ng/pl) was injected to rat CA1 area and MeCP2 SUMOylation was determined 1 h later (n=6 each group; t_{1,10}=0.57, P>0.05). Dex: dexamethasone. One-way ANOVA followed by post-hoc Newman-Keul multiple comparisons (a and b) or Student's t-test (c-e). Data are expressed as mean±SEM.

FIGS. 4(a)-4(e) SUMOylation of MeCP2 decreases its interaction with CREB and increases CREB DNA binding and Bdnf gene expression and increases methyl-DNA binding. (a) Flag-vector, Flag-MeCP2WT, Flag-MeCP2K412R or Flag-MeCP2WT-SUMO1 fusion plasmid was transfected to rat CA1 area and the association between MeCP2 and CREB was examined by co-IP using anti-Flag antibody and anti-CREB antibody. Plasmid transfection and expression was confirmed by western blot using anti-Flag antibody (left panel). Cell lysates were also immunoprecipitated with anti-MeCP2 antibody and immunoblotted with anti-SUMO1 antibody showing the specific band of sumoylated MeCP2 (middle panel). The quantified result of co-IP is shown in the right panel (n=3 each group; F_{3,8}=72.69, #P<0.001; q=11.99, #P<0.001, Flag-MeCP2K412R group vs Flag-MeCP2WT group; q=4.31, *P<0.05, Flag-MeCP2WT-SUMO1 group vs Flag-MeCP2WT group. (b) The same plasmids were transfected to rat CA1 area and CREB DNA binding activity was determined by oligo pull-down assay. CREB expression was determined by western blot. Plasmid transfection and expression was confirmed by western blot using anti-Flag antibody (n=7 each group; F_{3,24}=52.07, #P <0.001; q=3.26, *P <0.05, Flag-MeCP2WT group vs Flag-vector group; q=8.94, #P<0.001, Flag-MeCP2K412R group vs Flag-vector group; q=8.2, #P<0.001, Flag-MeCP2WT-SUMO1 group vs Flag-vector group; q=12.21, #P<0.001, Flag-MeCP2K412R group vs Flag-MeCP2WT group; q=4.93, *P<0.05, Flag-MeCP2WT-SUMO1 group vs Flag-MeCP2WT group; q=17.14, #P<0.001, Flag-MeCP2WT-SUMO1 group vs Flag-MeCP2K412R group. (c) The same plasmids were transfected to rat CA1 area and Bdnf mRNA level was determined by RT-qPCR and quantified over the Gapdh mRNA level (n=6 each group; F_{3,20}=38.45, #P<0.001; q=7.26, #P<0.001, Flag-MeCP2K412R group vs Flag-vector group; q=7.56, #P<0.001, Flag-MeCP2WT-SUMO1 group vs Flag-vector group. (d) The same plasmids were transfected to rat CA1 area and direct CREB binding to the Bdnf promoter was determined by ChIP assay. Plasmid transfection and expression was confirmed by western blot using anti-Flag antibody. (e) V5-MeCP2T158M, V5-MeCP2WT alone or together with the Flag-PIAS1 and Myc-SUMO1 plasmids were co-transfected to HEK293T cells and cell lysates were subject to methyl-DNA binding assay and in vitro SUMOylation assay (n=3 each group; F_{3,8}=230.02, #P<0.001; q=11.53, #P<0.001, V5-MeCP2T158M group vs V5-MeCP2WT group; q=15.75, #P<0.001, Flag-PIAS1+Myc-SUMO1+V5-MeCP2WT group vs V5-MeCP2WT group). One-way ANOVA followed by post-hoc Newman-Keul multiple comparisons. Data are expressed as mean±SEM.

FIGS. 5(a)-5(e) Several MECP2 mutations identified in RTT patients show decreased level of MeCP2 SUMOylation and decreased interaction with PIAS1. (a) V5-MeCP2WT plasmid or different V5-MeCP2 mutant plasmids associated with RTT was co-transfected with Flag-PIAS1 and Myc-SUMO1 plasmids to HEK293T cells and cell lysates were subject to in vitro SUMOylation assay. The level of MeCP2 phosphorylation at Ser-421 was examined using the phospho-Ser421 MeCP2 antibody.

The lower band in lane 8 indicates the truncated MeCP2R168X protein. (b) The quantified result of MeCP2 SUMOylation is shown (n=4 each group; F_{9,30}=579.1, #P<0.001; q=57.8, ^{#}P<0.001, V5-MeCP2R106W group vs V5-MeCP2WT group; q=41.81, #P<0.001, V5-MeCP2R133C group vs V5-MeCP2WT group; q=16.69, #P<0.001, V5-MeCP2P152A group vs V5-MeCP2WT group; q=58.68, #P<0.001, V5-MeCP2T158M group vs V5-MeCP2WT group; q=46.2, #P<0.001, V5-MeCP2R306C group vs V5-MeCP2WT group; q=33.56, #P<0.001, V5-MeCP2P376R group vs V5-MeCP2WT group, one-way ANOVA followed by post-hoc Newman-Keul multiple comparisons). (c) The quantified result of MeCP2 phosphorylation at Ser-421 is shown (n=3 each group; F_{9,20}=119.55, #P<0.001; q=16.73, #P<0.001, V5-MeCP2R106W group vs V5-MeCP2WT group; q=14.48, #P<0.001, V5-MeCP2R133C group vs V5-MeCP2WT group; q=3.54, *P<0.05, V5-MeCP2P152A group vs V5-MeCP2WT group; q=16.63, #P<0.001, V5-MeCP2T158M group vs V5-MeCP2WT group; q=4.97, P <0.01, V5-MeCP2R306C group vs V5-MeCP2WT group; q=4.52, P<0.01, V5-MeCP2P376R group vs V5-MeCP2WT group, one-way ANOVA followed by post-hoc Newman-Keul multiple comparisons). (d) V5-MeCP2WT plasmid or V5-tagged individual MeCP2 mutant plasmid was transfected to HEK293T cells and co-IP experiments were carried out with immunoprecipitation using anti-V5 antibody and immunoblotting using anti-Flag antibody. The expression level of MeCP2 (WT or mutant proteins) was examined by western blot using anti-VS antibody. (e) The quantified result of the association between PIAS1 and MeCP2 (or MeCP2 mutant proteins) is shown (n=3 each group; F_{7,16}=27.47, #P<0.001; q=5.85, **P<0.01, V5-MeCP2R106W group vs V5-MeCP2WT group; q=3.01, *P=0.05, V5-MeCP2R133C group vs V5-MeCP2WT group; q=8.46, #P<0.001, V5-MeCP2P152A group vs V5-MeCP2WT group; q=5.60, **P <0.01, V5-MeCP2T158M group vs V5-MeCP2WT group; q=11.09, #P<0.001, V5-MeCP2R306C group vs V5-MeCP2WT group; q=10.88, #P<0.001, V5-MeCP2P376R group vs V5-MeCP2WT group, one-way ANOVA followed by post-hoc Newman-Keul multiple comparisons). Data are expressed as mean±SEM.

FIGS. 6(a)-6(h) SUMOylation of MeCP2 rescues Mecp2 cKO mice-induced behavioral and LTP deficits. Mecp2 loxp mice and Mecp2 cKO mice transducted with different MeCP2 lenti-mRFP-vectors in their BLA area were subject to (a) motor activity test, (b) social ability test, and (c) social novelty test 7-10 days later (n=10-11 each group; F_{4,47}=19.87, #P<0.001 for sniffing to right stranger 2; q=8.55, Mecp2 cKO+lenti-mRFP-vector group vs Mecp2 loxp+lenti-mRFP-vector group; q=7.85, Mecp2 cKO+lenti-mRFP-MeCP2WT group vs Mecp2 cKO+lenti-mRFP-vector group; q=6.56, Mecp2 cKO+lenti-mRFP-MeCP2K412R group vs Mecp2 cKO+lenti-mRFP-MeCP2WT group; q=8.29, Mecp2 cKO+lenti-mRFP-MeCP2WT-SUMO1 group vs Mecp2 cKO+lenti-mRFP-MeCP2K412R group). (d) The same mice were subject to cued fear conditioning learning 7 days later (F_{4,47}=37.17, #P<0.001; q=10.77, Mecp2 cKO+lenti-mRFP-vector group vs Mecp2 loxp+lenti-mRFP-vector group; q=5.27, Mecp2 cKO+lenti-mRFP-MeCP2K412R group vs Mecp2 loxp+lenti-mRFP-vector group; q=5.75, Mecp2 cKO+lenti-mRFP-MeCP2K412R group vs Mecp2 cKO+lenti-mRFP-vector group; q=4.07, Mecp2 cKO+lenti-mRFP-MeCP2WT-SUMO1 group vs Mecp2 loxp+lenti-mRFP-vector group). (e) The BLA tissue from Mecp2 loxp+lenti-mRFP-vector and Mecp2 cKO+lenti-mRFP-vector groups of mice were subject to western blot analysis of MeCP2 expression (t_{1,18}=20.99, #P<0.001). (f) The BLA tissue of mice from Mecp2 cKO+lenti-mRFP-vector , Mecp2 cKO+lenti-mRFP-MeCP2WT and Mecp2 cKO+lenti-mRFP-MeCP2K412R groups were subject to western blot analysis of MeCP2 expression and MeCP2 phosphorylation at Ser-421 (For MeCP2 expression, F_{2,28}=241.64, #P<0.001; q=27.46, Mecp2 cKO+lenti-mRFP-MeCP2WT group vs Mecp2 loxp+lenti-mRFP-vector group; q=26.58, Mecp2 cKO+lenti-mRFP-MeCP2K412R group vs Mecp2 loxp+lenti-mRFP-vector group. For pS421MeCP2, F_{2,28}=3.72, *P<0.05; q=3.59, Mecp2 cKO+lenti-mRFP-MeCP2WT group vs Mecp2 loxp+lenti-mRFP-vector group; q=3.04, Mecp2 cKO+lenti-mRFP-MeCP2K412R group vs Mecp2 loxp+lenti-mRFP-vector group). (g) Aged female Mecp2 cKO mice transducted with different MeCP2 lenti-mRFP-vectors in their CA1 area were subject to LTP recording 7 days later under HFS paradigm. The Mecp2 loxp mice received lenti-mRFP vector transduction (n=5 each group; F_{3,16}=14.36, #P<0.001; q=6.3, Mecp2 cKO group vs Mecp2 loxp group; q=8.45, Mecp2 cKO+MeCP2WT group vs Mecp2 cKO group; q=3.31, Mecp2 cKO+MeCP2K412R group vs Mecp2 cKO group for the first 10 min recording. (h) The same groups of mice were subject to LTP recording under TBS paradigm (n=5 each group; F_{3,16}=14.65, #P<0.001; q=7.08, Mecp2 cKO group vs Mecp2 loxp group; q=8.05, Mecp2 cKO+MeCP2WT group vs Mecp2 cKO group; q=4.69, Mecp2 cKO+MeCP2K412R group vs Mecp2 cKO group for the first 10 min recording. Arrow indicates delivery of HFS or TBS. One-way or two-way ANOVA and Newman-Keul statistics. *P<0.05 and #P<0.001. Data are expressed as mean±SEM.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. DEFINITIONS

For convenience, certain terms employed in the context of the present disclosure are collected here. Unless defined otherwise, all technical and scientific terms used herein have the same meaning as commonly understood by one of the ordinary skill in the art to which this invention belongs.

The term “neurodevelopmental disorder” refers to any condition, disease, disorder characterized by abnormal neurodevelopment and/or basic behavioral processes, including attentional and perceptual processing, executive function, inhibitory control (e.g., sensory gating), social cognition, and communication and affiliative behaviors. Exemplified neurodevelopmental disorders include, but are not limited to, attention deficit hyperactivity disorder (ADHD), schizophrenia, obsessive-compulsive disorder (OCD), mental retardation, autistic spectrum disorders, cerebral palsy, articulation disorder, Rett syndrome, and learning disabilities (i.e., reading or arithmetic). In some embodiments, the neurodevelopmental disorder is Rett syndrome, which is an autistic spectrum disorder caused by mutations of the MeCP2 gene.

The term “Rett syndrom” refer to a spectrum of neurodevelopmental disorders characterized by impaired social interaction and communication accompanied by repetitive and stereotyped behavior. Autism includes a spectrum of impaired social interaction and communication, however, the disorder can be roughly categorized into “high functioning autism” or “low functioning autism,” depending on the extent of social interaction and communication impairment. Individuals diagnosed with “high functioning autism” have minimal but identifiable social interaction and communication impairments (i.e., Asperger's syndrome).

The term “expression” as used herein is intended to refer to transcription of a gene when a condition is met, resulting in the generation of mRNA and usually encoded protein. Expression can be achieved or performed naturally by the cell (i.e., without artificially intervention) or may be achieved or performed artificially (i.e., with the io involvement of artificially intervention, such as by the use of promoters regulated by the use of a chemical agent). The expression may also be initiated by a recombination event triggered by a site-specific recombinase, such as by Cre-mediated recombination. Expression may be determined by measuring mRNA transcribed from the gene or by measuring protein encoded by the gene.

The term “nucleic acid” refers to polynucleotides such as deoxyribonucleic acid (DNA) and where appropriate, ribonucleic acid (RNA). Nucleic acids include but are not limited to single-stranded and double-stranded polynucleotides. Illustrative polynucleotides include DNA, single-stranded DNA, cDNA, and mRNA. The term also includes, analogs of either DNA or RNA made from nucleotide analogs, and as applicable, single (sense or antisense) and double-stranded polynucleotides. The term further includes modified polynucleotides, including modified DNA and modified RNA, e.g., DNA and RNA comprising one or more unnatural nucleotide or nucleoside. The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, and/or which have similar binding properties as the reference nucleic acid, and/or which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also encompasses conservatively modified variants thereof (e.g., degenerate codon substitutions) and complementary sequences, as well as the sequence explicitly indicated.

The term “vector” refers to a nucleic acid molecule capable of transporting another nucleic acid to which it has been linked. The term “expression vector” refers to a vector comprising a promoter operably linked to a nucleic acid in a manner allowing expression of the operably linked nucleic acid. Vectors or expression vectors as used herein thus include plasm ids or phages capable of synthesizing the subject protein encoded by the respective recombinant gene carried by the vector. Vectors or expression vectors also include viral-based vectors capable of introducing a nucleic acid into a cell, e.g., a mammalian cell. Certain vectors are capable of autonomous replication and/or expression of nucleic acids to which they are linked.

In the present disclosure, nucleic acid is “operably linked” when it is placed into a functional relationship with another nucleic acid sequence. In general, “operably linked” means that the DNA sequences being linked are contiguous, and in reading phase. However, enhancers do not have to be contiguous. Linking is accomplished by ligation at convenient restriction sites, if such sites do not exist, the synthetic oligonucleotide adaptors or linkers are used in accordance with conventional practice.

The term “transduction” refers to the process of deliberately introducing nucleic acid into cells, preferably animal cells, by use of a viral vector. Suitable viral vectors that may be used in the present disclosure are those derived from herpes virus, retrovirus, vaccinia virus, attenuated vaccinia virus, canary pox virus, adenovirus, or adeno-associated virus.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

The term “treatment” as used herein are intended to mean obtaining a desired pharmacological and/or physiologic effect, e.g., delaying or inhibiting platelet aggregation and/or platelet activation. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, intraveneously, intramuscularly, intraperitoneally, intraarterially, intracranially, or subcutaneously administering an agent (e.g., a modified MeCP2 or a nucleic acid encoding the modified MeCP2) of the present invention. In some embodiments, the modified MeCP2 or the nucleic acid encoding the modified MeCP2 is formulated into powders for mixed with suitable carrier (e.g., buffer solution) before use, such as intraveneous injection.

The term “an effective amount” as used herein refers to an amount effective, at dosages, and for periods of time necessary, to achieve the desired result with respect to the treatment of a disease resulted from platelet aggregation. For example, in the treatment of a thrombotic disorder, an agent (i.e., the modified MeCP2 or the nucleic acid encoding the modified MeCP2) which decrease, prevents, delays or suppresses or arrests any symptoms of the thrombotic disorder would be effective. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) or a ratio of mass of the active agent to body mass, e.g., as milligrams per kilogram (mg/kg). The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

The term “pharmaceutically acceptable” refers to molecules and compositions that do not produce an adverse or undesirable reaction (e.g., toxicity, or allergic reaction) when administered to a subject, such as a human.

The term “excipient” as used herein means any inert substance (such as a powder or liquid) that forms a vehicle/carrier for the active agent. The excipient is generally safe, non-toxic, and in a broad sense, may also include any known substance in the pharmaceutical industry useful for preparing pharmaceutical compositions such as, fillers, diluents, agglutinants, binders, lubricating agents, glidants, stabilizer, colorants, wetting agents, disintegrants, and etc.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1% , or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. DETAIL DESCRIPTION OF PREFERRED EMBODIMENTS

The present disclosure is based, at least in part, on the unexpected discovery that expression of a wild type MeCP2 polypeptide or a modified MeCP2 polypeptide in a Mecp2 conditional knockout mouse (cko) may rescue the deficits of social interaction, fear memory, and long-term potentiation (LTP) associated with neurodevelopmental disorders and/or conditions. Accordingly, the wild-type or modified MeCP2 polypeptide may serve as a potential drug candidate for the development of a medicament for the treatment or prophylaxis of neurodevelopmental disorders.

The practices of this invention are hereinafter described in detail with respect to use of a MeCP2 polypeptide, the preparation of a medicament for preventing or treating a subject or patient who suffers from a neurodevelopmental disorder.

As shown in the accompanying Examples, it has been unexpectedly demonstrated that the expression of a wild type MeCP2 or a modified MeCP2 may rescue the deficits of social interaction, fear memory, and long-term potentiation (LTP) in a Mecp2 cko animal. Accordingly, one important aspect of the present disclosure is to provide a method of treating a subject suffering from a neurodevelopmental disorder by introducing a wild type MeCP2 or a modified MeCP2 to the subject. Without wishing to be bound to any particular theory, it is believed that modification of the MeCP2 polypeptide by sumoylation, phosphorylation or both, alters its activity in a manner resulting in improved social interaction, memory performance, as well as synaptic plasticity in the subject.

According to embodiments of the present disclosure, the MeCP2 may be a wild type polypeptide or has post-translational modification, e.g., phosphorylation or sumoylation. Particular embodiments contemplate that the human MeCP2 polypeptide is post-translationally modified by phosphorylation at a single site. Certain embodiments contemplate that the MeCP2 polypeptide is phosphorylated. Particular embodiments contemplate that MeCP2 polypeptide is post-translationally modified by sumoylation at a single site, and that the E3 ligase PIAS1 plays a critical role in this process. Certain embodiments contemplate that the MeCP2 polypeptide is sumolyated.

“Phosphorylation,” as used herein, refers to the addition of a phosphate (PO₄³⁻) group to an amino acid residue of a polypeptide. Reversible protein phosphorylation, principally on serine, threonine or tyrosine residues, is one of the most important and well-studied post-translational modifications. Phosphorylation plays critical roles in the regulation of many cellular processes including cell cycle, growth, apoptosis and signal transduction pathways. In certain embodiments, the MeCP2 polypeptide is a modified MeCP2 polypeptide comprising an amino acid modification, in which threonine at position 308 of the wild type MeCP2 polypeptide is phosphorylated. In some embodiments, the MeCP2 polypeptide is a modified MeCP2 polypeptide comprising an amino acid modification, in which serine at position 421 of the wild type MeCP2 polypeptide is phosphorylated.

“Sumoylation or SUMOylation” as used herein refers to the covalent linkage of a polypeptide to the SUMO protein (Small Ubiquitin-related Modifier). SUMO proteins are a family of small proteins that are covalently attached to and detached from other proteins in cells to modify their function. Sumoylation is a post-translational modification involved in various cellular processes, such as nuclear-cytosolic transport, transcriptional regulation, apoptosis, protein stability, response to stress, and progression through the cell cycle. In certain embodiments, the MeCP2 polypeptide is a modified MeCP2 polypeptide comprising an amino acid modification, in which lysine at position 412 of the wild type MeCP2 polypeptide is sumolayted. According to certain embodiments of the present disclosure, phosphorylation of the MeCP2 polypeptide at Ser308 or Thr421 results in increased level of MeCP2 sumoylation. By contrast, phosphorylation at other sites, such as Ser80, Ser164, Ser229 and Ser399, does not alter the level of MeCP2 sumoylation.

The MeCP2 may be provided to the subject in need thereof by administering the MeCP2 polypeptide or a nucleic acid encoding the MeCP2 polypeptide to the subject. In some embodiments, the MeCP2 polypeptide is a wild type human MeCP2 protein. In other embodiments, the MeCP2 polypeptide is a modified human MeCP2 polypeptide comprising one or more amino acid modifications. In various embodiments, the one or more amino acid modifications comprises a modification at an amino acid position by sumoylation or phosphorylation of the wild-type MeCP2 polypeptide, such as a modification of an amino acid at position 308 of the full length human wild-type MeCp2 polypeptide, e.g., phosphorylation of Ser, or a modification of an amino acid at position 421 of the full length human wild-type MeCP2 polypeptide, e.g., phosphorylation of Thr, or a modification of an amino acid at position 412 of the full length human wild-type MeCP2 polypeptide, e.g., sumoylation of Lys.

One method includes administering to the subject an effective amount of a polypeptide or a nucleic acid encoding the polypeptide, wherein the polypeptide is a modified human MeCP2, in which the lysine residue at position 412 is sumoylated.

Another method includes administering to the subject an effective amount of a polypeptide or a nucleic acid encoding the polypeptide, wherein the polypeptide is a modified human MeCP2, in which the threonine residue at position 308 or the serine residue at position 421, is phosphorylated.

The present polypeptide can be synthesized using methods known in the art or be prepared using recombinant technology. For example, one can clone a nucleic acid encoding the MeCP2 polypeptide (e.g., the wild type MeCP2, or the modified MeCP2) in an expression vector, in which the nucleic acid is operably linked to a regulatory sequence suitable for expressing the polypeptide in a host cell. One can then introduce the vector into a suitable host cell to express the polypeptide. The expressed recombinant polypeptide can be purified from the host cell by methods such as ammonium sulfate precipitation and fractionation column chromatography. A polypeptide thus prepared can be tested for its activity according to the methods described in the art or herein.

The present polypeptides may be administered to a subject in a variety of manner, including as “naked” polypeptides or complexed with a delivery-enhancing agent. In particular embodiments, the MeCP2 polypeptide is associated with an agent that enhances cellular uptake.

The nucleic acid encoding the MeCP2 polypeptide can be delivered by the use of polymeric, biodegradable microparticle or microcapsule delivery devices known in the art. Another way to achieve uptake of the nucleic acid in a host is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells. Alternatively, tissue specific targeting can be achieved by the use of tissue-specific transcriptional regulatory elements that are known in the art.

Delivery of “naked DNA” (i.e., without a delivery vehicle) to an intramuscular, intradermal, or subcutaneous site is another means to achieve in vivo expression.

Nucleic acids may be administered to a subject in a variety of manners, including as “naked” DNA or RNA, or complexed with lipids or encapsulated in a lipid particle. In particular embodiments wherein a nucleic acid, i.e., a polynucleotide, is administered to the subject, the nucleic acid is present in an expression vector. In certain embodiments, the nucleic acid is present in a viral vector. The viral vector may be a replication defective or replication competent viral vector. In various embodiments, the viral vector is derived from a herpes virus, a retrovirus, a lentivirus, a vaccinia virus, an attenuated vaccinia virus, a canary pox virus, an adenovirus, or an adeno-associated virus. The nucleic acid may be present in an expression vector, in which the nucleic acid sequence encoding a MeCP2 polypeptide is operatively linked to a promoter or enhancer-promoter combination. Suitable expression vectors include plasmids and viral vectors such as herpes viruses, retroviruses, vaccinia viruses, attenuated vaccinia viruses, canary pox viruses, adenoviruses and adeno-associated viruses. Particular embodiments contemplate that the nucleic acids are present in vectors derived from retroviruses, e.g., lentiviral vectors. In particular embodiments, the nucleic acid is operably linked to a promoter sequence and, optionally enhancer elements. In particular embodiments, the promoter and/or enhancer elements confer tissue-specific expression of the nucleic acid and its encoded polypeptide.

In certain embodiments wherein the polypeptide or the nucleic acid is administered to the subject, the method further comprises administering to the subject an effective amount of another active agent that induces the sumolyation of the MeCP2 polypeptide. Suitable active agent that may be used along with the present method is N-methyl-D-aspartate (NMDA), an insulin-like growth factor (IGF-1) or corticotropin-releasing factor (CRF). In particular embodiments, the active agent is NMDA. In some embodiments, the active agent is IGF-1. In other embodiments, the active agent is CRF.

According to some embodiments of the present disclosure, the MeCP2 may be administered to the subject via intravascular delivery (e.g., injection or infusion), oral, enteral, rectal, pulmonary (e.g., inhalation), nasal, topical (including transdermal, buccal and sublingual), intravesical, intravitreal, intraperitoneal, vaginal, brain delivery (e.g., intracerebroventricular, and intracerebral), CNS delivery (e.g., intrathccal, perispinal, and intra-spinal), parenteral (e.g., subcutaneous, intramuscular, intravenous, and intradermal), or transmucosal delivery in the amount of 0.001-100 mg/Kg, preferably in the amount of 0.01-80 mg/Kg; more preferably in the amount of 0.1-50 mg/Kg.

The second aspect of the present application is directed to a medicament or a pharmaceutical composition for treating a neurodevelopmental disease, disorder and/or condition. The pharmaceutical composition comprises an effective amount of a MeCP2 polypeptide or a nucleic acid encoding the MeCP2 polypeptide; and a pharmaceutically acceptable excipient.

Particular embodiments contemplate that the MeCP2 polypeptide is a wild type MeCP2 polypeptide. Other particular embodiments contemplate that the MeCP2 polypeptide is a modified MeCP2 polypeptide comprising one or more amino acid modifications. In some embodiments, the modified MeCP2 polypeptide comprises one amino acid modification, in which the amino acid at position 308 of the full length human wild-type MeCp2 polypeptide is phosphorylated. In other embodiments, the modified MeCP2 polypeptide comprises one amino acid modification, in which the amino acid at position 421 of the full length human wild-type MeCP2 polypeptide is phosphorylated. In further embodiments, the modified MeCP2 polypeptide comprises one amino acid modification, in which the amino acid at position 412 of the full length human wild-type MeCP2 polypeptide is sumoylated.

Generally, the MeCP2 polypeptide is present in the pharmaceutical composition at a level of about 0.01% to 99.9% by weight, based on the total weight of the pharmaceutical composition. In some embodiments, the MeCP2 polypeptide is present at a level of at least 0.1% by weight, based on the total weight of the pharmaceutical composition. In certain embodiments, the MeCP2 polypeptide is present at a level of at least 5% by weight, based on the total weight of the pharmaceutical composition. In still other embodiments, the MeCP2 polypeptide is present at a level of at least 10% by weight, based on the total weight of the pharmaceutical composition. In still yet other embodiments, the MeCP2 polypeptide is present at a level of at least 25% by weight, based on the total weight of the pharmaceutical composition.

In some embodiments, the pharmaceutical composition of this invention further includes an active agent known to induce sumoylation of the MeCP2 polypeptide that facilitates alleviating or ameliorating symptoms of the neurodevelopmental disease, disorder, and/or condition. Examples of such active agent include, and are not limited to, N-methyl-D-aspartate (NMDA), an insulin-like growth factor (IGF-1) or corticotropin-releasing factor (CRF) In particular embodiments, the active agent is NMDA. In some embodiments, the active agent is IGF-1. In other embodiments, the active agent is CRF.

Pharmaceutically acceptable excipients are those that are compatible with other ingredients in the formulation and biologically acceptable.

The pharmaceutical composition may comprise different types of excipients depending on the intended routes of administration. The present composition may be administered intraveneously, intradermally, intraarterially, intraperitoneally, intralesionally, intracranially, intranasally, intrapleurally, intratracheally, intrarectally, topically, intramuscularly, subcutaneoustly, intravesicularlly, intrapericardially, intraocularally, orally, topically, locally, injection, inhalation, infusion, localized perfusion, in any suitable forms such as powders, creams, liquids, aerosols and etc.

The actual dosage of the medicament or the pharmaceutical composition may be determined by the attending physician based on the physical and physiological factors of the subject, these factors include, but are not limited to, age, gender, body weight, the disease to be treated, severity of the condition, previous history, the presence of other medications, the route of administration and etc. According to non-limiting examples of the present disclosure, each dosage will give rise to 0.001-100 mg MeCP2 polypeptide/Kg body weight per administration.

The pharmaceutical compositions containing the MeCP2 polypeptide may be in a form suitable for oral use, for example, as tablets, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain MeCP2 polypeptide in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

The tablets may be uncoated or they may be coated by known techniques to delay disintegration and absorption in the gastrointestinal tract and thereby provide a sustained action over a longer period.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

For example, a solid oral composition such as a tablet or capsule may contain from 1 to 99% (w/w) MeCP2 polypeptide; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment.

A parenteral formulation (such as a solution or suspension for injection or a solution for infusion) may contain from 1 to 50% (w/w) MeCP2 polypeptide; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilizers, tonicity adjusting agents and preservatives.

The pharmaceutical compositions of the invention may be in the form of an oil-in-water emulsion. The oily phase may be a vegetable oil, for example olive oil or peanut oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a preservative, and flavouring and colouring agents. The pharmaceutical compositions may be in the form of a sterile injectable aqueous or oleagenous suspension. This suspension may be formulated according to the known art using suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringers solution and isotonic sodium chloride solution. Co-solvents such as ethanol, propylene glycol or polyethylene glycols may also be used. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The MeCP2 polypeptide may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug.

Such materials are cocoa butter and polyethylene glycols.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

### Materials and Methods

### Animals.

Adult male (2-3 months) and aged female (6-8 months) Mecp2^{Ioxp} mice were used in this study. They were purchased from Jackson Laboratory (Bar Harbor, Me., USA) (strain name: B6.129P2-Mecp2tm1Bird/J, stock number: 006847), bred and mated at the Animal Facility of the Institute of Biomedical Sciences (IBMS), Academia Sinica in Taiwan (R. 0. C.). Adult male Sprague-Dawley rats (250-350 g), bred at the Animal Facility of IBMS, were also used in the present study. All the animals were housed and maintained on a 12/12 hr light/dark cycle (light on at 6:30 am) with access to food and water ad libitum. Animals were randomly divided to different experimental groups. All experimental procedures were complied with the guidelines and ethical regulations of Animal Use and Care of the National Institute of Health and were approved by the Animal Committee of IBMS, Academia Sinica.

Hippocampal Lysate and Cell Lysate Preparation. Animals were killed by decapitation, and their hippocampal tissue was dissected out. Rat hippocampal tissue was lysed by brief sonication in lysis buffer containing 50 mM Tris-HCl (pH 7.4), 150 mM NaCl, 2 mM EDTA, 1% IGEPAL CA-630, 1 mM phenylmethylsulfonyl fluoride (PMSF), 20 pμg/ml pepstatin A, 20 μg/ml leupeptin, 20 μg/ml aprotinin, 50 mM NaF, and 1 mM Na₃VO₄. The primary neuron cell lysate and HEK293T cell lysate were prepared in 1 ml of lysis buffer containing 20 mM Tris (pH 7.4), 150 mM NaCl, 1 mM MgCl2, 1% IGEPAL CA-630, 10% glycerol, 1 mM dithiothreitol (DTT), 50 mM β-glycerophosphate, 50 mM NaF, 10 μg/ml PMSF, 4 μg/ml aprotinin, 4 μg/ml leupeptin, and 4 μg/ml pepstatin.

### Immunoprecipitation (IP) and Western Blot.

The E3 SUMO-protein ligase PIAS1, MeCP2, Flag, V5 and Myc, the clarified lysate (0.5 mg) was immunoprecipitated by mixed with 0.5 μl of anti-PIAS1 antibody (Catalog No. 2474-1, Epitomics, Burlingame, Calif.), 2 μl of anti-MeCP2 antibody (Catalog No. 3456, Cell Signaling, Danvers, Mass.), 2 μl of anti-Flag M2 antibody (Catalog No. F1804, Sigma-Aldrich, St. Louis, Mo.), 1 μl of anti-VS antibody (Catalog No. MCA2895, AbD Serotec, Kidlington, UK) and 2 μl of anti-Myc antibody (Catalog No. 05-419, Millipore, Bedford, Mass.) at 4° C., and allowed the mixture to react overnight. Twenty microliter of rabbit or mouse IgG was used in the control group. The protein A or G magnetic beads (30 ml, 50% slurry, GE Healthcare, Barrington, IL) were added to the IP reaction product to catch the immune complex at 4° C. for 3 h. The immune complex on beads were washed three times with washing buffer containing 20 mM HEPES (pH 7.4), 150 mM NaCl, 1 mM EDTA, 1% IGEPAL CA-630, 1 mM DTT, 50 mM β-glycerophosphate, 50 mM NaF, 10 mg/ml PMSF, 4 pg/ml aprotinin, 4 μg/ml leupeptin and 4 μg/ml pepstatin, and then subject to 8% SDS-PAGE followed by transferring onto the Nitrocellulose (NC) membrane (GE Healthcare). Western blot was conducted using the following antibodies: rabbit anti-PIAS1 (1:10000, Catalog No. 2474-1, Epitomics), anti-MeCP2 (1:2000, Catalog No. 3456, Cell Signaling), anti-phospho-Ser421MeCP2 (1:1000, Catalog No. AP3693a, ABGENT, San Diego, Calif.), anti-SUMO1 (1:4000, Catalog No. 40120 SUMOlink kit, Active Motif, Carlsbad, Calif.), anti-ubiquitin (1:3000, Catalog No. 3936, Cell Signaling), anti-Flag M2 (1:5000, Catalog No. F1804, Sigma-Aldrich), anti-V5 (1:8000, Catalog No. MCA2895, AbD Serotec), anti-His (1:5000, Catalog No. 0B05, Millipore, Bedford, Mass.), anti-GST (1:5000, Catalog No. 110736, GeneTex, San Antonio, Tex.), anti-RFP (1:2000, Catalog No. 600-401-379, Rockland, Gilbertsville, Pa.) and anti-actin (1:200000, Catalog No. MAB1501, Millipore). The secondary antibody used was HRP-conjugated goat-anti rabbit IgG antibody or goat-anti mouse IgG antibody (Chemicon). Membrane was developed by reacting with chemiluminescence HRP substrate (Millipore) and exposed to the LAS-3000 image system (Fujifilm, Tokyo, Japan) for visualization of protein bands. The protein bands were quantified by using the NIH Image J Software.

### Plasmid Construction and DNA Transfection.

For the construction of the V5-tagged MeCP2 plasmid, full-length Mecp2 was cloned by amplifying the rat hippocampal Mecp2 cDNA (accession # NM_022673) with primers 5′-ATTTGCGGCCGCCACCATGGTAGCTGGGATGTTAG-3′ (SEQ ID NO: 1) and 5′-TAACCGCGGGCTAACTCTCTCGGTCAC-3′ (SEQ ID NO: 2). The PCR product was sub-cloned between the Notl and SacII sites of the mammalian expression vector pcDNA3.1-V5-His. Mecp2 mutant plasmids were generated using the QuickChange Site-Directed Mutagenesis Kit (Stratagene, La Jolla, Calif.). For the construction of the mRFP-tagged Mecp2 plasmid, wild-type Mecp2, Mecp2K412R, Mecp2WT-SUMO1 and Mecp2WT-SUMO1 fusion plasm ids were sub-cloned into the pmRFP expression vector (Addgene plasmid #13990). For the construction of the Flag-tagged Pias1 plasmid, the procedure used was the same as that described previously (Tai et al., EMBO J. 30. 205-220 (2011)). HEK293T cells and Neuro2A cells were maintained in Dulbecco's modified Eagle's medium (DMEM) containing 10% fetal bovine serum and incubated at 37° C. in a humidified atmosphere with 5% CO₂ as described previously (Tai et al., I. Biol. Chem. 284, 4073-4089 (2009)). Transfection was made by using the Lipofectamine 2000 reagent (Invitrogen, Carlsbad, Calif.) in 12-well culture plates according to the manufacturer's instructions.

### Lentiviral Vector Construction and Preparation.

For the construction of mRFP, mRFP-Mecp2WT, mRFP-Mecp2K412R and mRFP-Mecp2WT-SUMO1 lentivitral vectors, full-length mRFP-MeCP2WT, mRFP-MeCP2K412R and mRFP-MeCP2WT-SUMO1 fusion plasmids were sub-cloned into the lentiviral vector pLenti-Tri-cistronic (ABM, Richmond, BC, Canada) by amplifying different pmRFP-MeCP2 related non-viral constructs with different primers. The forward primers used for these five constructs were the same, and was 5′-ATCGGGATCCGCCACCATGGCCTCCTCCGAGGAC-3′ (SEQ ID NO: 3). The reverse primer for mRFP vector was:

5′-ATCGCCTAGGTTAGGCGCCGGTGGAGTGGCG-3′ (SEQ ID NO: 4) and that for mRFP-Mecp2WT or mRFP-Mecp2K412R was 5′-ATCGCCTAGGTCAGCTAACTCTCTCGGTCACGGG-3′ (SEQ ID NO: 5). The reverse primer used for mRFP-Mecp2-SUMO1 or mRFP-Mecp2WT-SUMO1 was: 5′-ATCGCCTAGGCTAAACCGTCGAGTGACCCCCCG-3′ (SEQ ID NO: 6) These PCR products were sub-cloned between BamHl and Avril sites of the lentiviral vector. For construction of GFP-2A-NLS-Cre lentivitral vector, full-length Cre recombinase cDNA was added with nuclear localization signal (NLS) by PCR-amplification and cloned into pLenti-Tri-cistronic (ABM) to obtain a bicistronic vector expressing both GFP and NLS-Cre. The primers used for Cre vector were:

5′ -ATC G GAATTC C CAAAGAAGAAGAGAAAG GTTATGTC CAATTTACTGAC C -3′ (forward, SEQ ID NO: 7) and 5′-ATCGGCGGCCGCCTAATCGCCATCTTCCAG-3′ (reverse, SEQ ID NO: 8). The PCR product was sub-cloned between the EcoRl and Notl sites of the lentiviral vector pLenti-Tri-cistronic (ABM). The GFP construct was cloned by amplifying the GFP gene from pLenti-CMV-GFP-2A-Puro-Blank (ABM) and sub-cloned into the pLenti-Tri-cistronic vector between Scal and Kpnl sites, upstream of the 2A peptide (a self-processing viral peptide bridge) and Nls-Cre sequences. The primers used for GFP vector were 5′-ATCGAGTACTGCCACCATGGAGATCGAGTGCCGCATC-3′ (forward, SEQ ID NO: 9) and 5′-ATCGGGTACCGGCGAAGGCGATGGGGGTC-3′ (reverse, SEQ ID NO: 10). For lentivirus packaging, HEK293LTV cells (Cell Biolabs, San Diego, Calif.) were transfected with 1.5 μg of psPAX2 (Addgene plasmid #12260), 0.5 μg of pMD2.G (Addgene plasmid #12259), and 2 μg of pLenti-GFP-2A-Nls-Cre, mRFP, mRFP-Mecp2, mRFP-Mecp2K412R, mRFP-Mecp2WT-SUMO1 or 2 μg of pLenti-CMV-GFP-2A-Puro-Blank (ABM) coding for GFP as control using 10 μl of Lipofectamine 2000 (Invitrogen) in 6-well cell culture dish. Lentiviral particles were collected using the speedy lentivirus purification solution (ABM) according to the manufacturer's protocols. Cell culture medium containing lentiviral particles can be harvested for two to three times at 12 h interval until 36 h after transfection, and it was kept at 4° C. for the collecting period. The collected culture medium was further clarified by centrifugation at 2,500×g for 10 min and filtrated through a 0.45 μm syringe filter. The speedy lentivirus purification solution (ABM) was added into filtrated supernatant (1:9, v/v) containing lentiviral particles and mixed thoroughly by inversion. The lentiviral supernatant was centrifuged at 5,000×g at 4° C. for 10 min. Supernatant was then discarded and the viral pellet was re-suspended in ice cold PBS. After titration, the viral stock was stored at −80° C. in aliquots. The lentivirus titer was determined by lentivirus qPCR Titer Kit (ABM) according to the manufacturer's protocols (ABM).

In vitro SUMOylation Assay.

In vitro SUMOylation assay was performed by using the SUMO link kit according to the manufacturer's instructions (Active Motif, Carlsbad, Calif.). Recombinant GST-tagged MeCP2 protein (Prospec, East Brunswick, N.J.), His-tagged PIAS1 protein purified from His-bind resin (Novagen, Wis.), and GST-tagged SENP1 protein (Enzo, Ann Arbor, Mich.) were used for assays at 30° C. for 3 h and then boiled in Laemmli sample buffer at 95° C. for 10 min. The in vitro SUMOylation product was subject to 8% SDS-PAGE followed by transferring onto the NC membrane. The membrane was immunoblotted with anti-His and anti-GST antibodies.

### In Vitro SUMOylation Assay for CA1 Tissue.

Hippocampal CA1 tissue lysate was prepared in the same way as that prepared for western blot. For immunoprecipitating the MeCP2, the clarified lysate (0.5 mg) was immunoprecipitated with 3 μl of anti-MeCP2 antibody (Catalog No. 3456, Cell Signaling) at 41° C. overnight. The protein A agarose beads (30 ml, 50% slurry, GE Healthcare, Barrington, Ill.) were added to the IP reaction product to catch the immune complex at 41° C. for 3 h. The immune complex on beads were washed three times with washing buffer containing 20 mM HEPES (pH 7.4), 150 mM NaCl, 1 mM EDTA, 1% IGEPAL CA-630, 1 mM DTT, 50 mM β-glycerophosphate, 50 mM NaF, 10 mg/ml PMSF, 4 mg/ml aprotinin, 4 mg/ml leupeptin and 4 mg/ml pepstatin and subject to in vitro SUMOylation reaction with the addition of recombinant PIAS1 protein (3 μl, Catalog No. BML-UW9960, Enzo Life

Sciences, Farmingdale, N.Y.), E1(1 μl), E2 (1 μl) and the SUMO1 (0.5 μl) proteins provided in the kit. In vitro SUMOylation assay was performed using the SUMO linkTM kit according to the manufacturer's instructions (Active Motif, Carlsbad, Calif.) and boiled in Laemmli sample buffer at 95° C. for 10 min. The in vitro SUMOylation product was subject to 8% SDS-PAGE followed by transferring onto the PVDF membrane (Millipore). The membrane was immunoblotted with anti-MeCP2 antibody (1:1000, Catalog No. 3456, Cell Signaling) or anti-SUMO1 antibody (1:4000, Catalog No. 40120, Active Motif). For determination of endogenous MeCP2 SUMOylation after PIAS1 siRNA transfection, no E1, E2, SUMO1 and PIAS1 proteins were added to the IP reaction product. The remaining procedures were the same as that for carrying out the in vitro SUMOylation assay.

### Pull-down Assay for MeCP2 Methyl-DNA Binding.

DNA oligonucleotides containing CpG element from the Bdnf gene (bold letter) (5′-CAATGCCCTGGAACGGAATTCTTCTAATAAAAGATGTATCATTTTAAATGC-3′, SEQ ID NO: 11) were conjugated with a 5′ biotin on the sense strand. DNA oligo sequence was synthesized at −125 base pairs from the transcription start site of Bdnf exon1. Both complementary oligonucleotides were annealed according to the procedure described previously (Tai et al., EMBO J. 30, 205-220 (2011)). The resulting DNA oligos were methylated with Sssl methylase (NEB, Ipswich, Mass.) according to the recommended protocol for methylation of DNA (NEB). For the MeCP2 methyl-DNA binding assay, the lysate (0.4 mg) from the HEK293T cell transfected with V5-MeCP2WT or V5-MeCP2T158M plasmid was added with 6 μl duplex oligonucleotides (100 μM) and poly dl-dC (1 μg/ml, GE Healthcare) at 4° C. for overnight. The streptavidin agarose beads (10 μl, Sigma) were added to the pull-down reaction product to catch the MeCP2-DNA oligonucleotide complex at 4° C. for 3 h. The pull-down reaction complex on beads were then washed three times with washing buffer containing 20 mM HEPES (pH 7.4), 150 mM NaCl, 1 mM EDTA, 1% IGEPAL CA-630, 1 mM DTT, 50 mM bglycerophosphate, 50 mM NaF, 10 pμg/ml PMSF, 4 μg/ml aprotinin, 4 μg/ml leupeptin and 4 μg/ml pepstatin and boiled in Laemmli sample buffer at 95° C. for 10 min. For the analysis of MeCP2 methyl-DNA binding activity, the pull-down assay product was subject to 8% SDS-PAGE followed by transferring onto the PVDF membrane (Millipore) and immunoblotted with anti-MeCP2 antibody (1:2000, Catalog No. 3456, Cell Signaling).

### Pull-Down Assay for CREB DNA-Binding Activity.

DNA oligonucleotides containing two CRE elements (bold letters) (sense strand: 5′-AGAGATTGCCTGACGTCAGAGAGCTAGGATTGCCTGACGTCAGAGAGCTAG-3′, SEQ ID NO: 12; and antisense strand: 5′-CTAGCTCTCTGACGTCAGGCAATCCTAGCTCTCTGACGTCAGGCAATCTCT-3′, SEQ ID NO: 13) were conjugated with a 5′ biotin on the sense strand according to the method described elsewhere (Vallejo et al., J. Biol. Chem. 267, 12876-12884 (1992)). Both complementary oligonucleotides were re-suspended in the annealing buffer (10 mM Tris [pH 8.0], 50 mM NaCl, 1 mM EDTA). For annealing the sense and anti-sense oligonucleotides, 10 μl each of the complementary oligonucleotides together with 80 μl of the annealing buffer were mixed in a 0.5 ml microtube and the tube was placed in a heating block at 90° C. The heating block was allowed to gradually cool down to room temperature and stored on ice or at −20° C. until use. For the CREB pull-down assay, the clarified hippocampal CA1 tissue lysate (0.4 mg) was added with 6 μl duplex oligonucleotides (100 μM) and poly dl-dC (1 μg/ml, GE Healthcare) at 4° C. for overnight. The streptavidin agarose beads (10 μl, Sigma-Aldrich) were added to the pull-down reaction product to catch the CREB-DNA oligonucleotide complex at 4° C. for 3 h. The pull-down reaction complex on beads was then washed three times with PBS and boiled in Laemmli sample buffer at 95° C. for 10 min. For analysis of CREB DNA-binding activity, the pull-down assay product was subject to 8% SDS-PAGE followed by transferring onto the PVDF membrane and immunoblotted with anti-CREB antibody (1:2000, Catalog No. 9197, Cell Signaling).

### Reverse Transcription-Quantitative Real-Time PCR (RT-qPCR).

Total RNA was isolated from 20 mg of hippocampal CA1 tissue using RNeasy Mini Kit (Qiagen, Germantown, Md.) according to the manufacturer's instructions. The RNA samples were re-suspended in nuclease-free water and quantified spectrophotometrically at 260 nm. All RNA samples had an A260:A280 value between 1.8 and 2.0. cDNA synthesis was carried out by using the QuantiTect Reverse Transcription Kit (Qiagen) according to the manufacturer's protocols. The cDNA stock was stored at −20° C. Quantitative PCR for Bdnf and the endogenous control gene glyceraldehyde 3-phosphate dehydrogenase (Gapdh) was carried out by using the iQ SYBR Green Supermix (Bio-rad). The primer sequences for Gapdh were: 5′- GGCAAGTTCAATGGCACAGT-3′ (forward, SEQ ID NO: 14) and 5′-TGGTGAAGACGCCAGTAGACTC-3′ (reverse, SEQ ID NO: 15). The primer sequences for Bdnf were: 5′-CTAGGACTGGAAGTGGAAA-3′ (forward, SEQ ID NO: 16) and 5′- ATTTCATGCTAGCTCGCCG-3′ (reverse, SEQ ID NO: 17). Amplification was performed by using the Rotor-Gene Q Real Time PCR system (Qiagen), and the reaction condition followed the manufacturer's protocols. The thermal cycler protocol used is as follows: 95° C. for 10 min, 95° C. for 10 s and 60° C. for 30 s (40 cycles). The cycle threshold (Ct) values and related data were analyzed by using the Rotor-Gene Q Real Time PCR System Software (Qiagen). The expression level of Bdnf was normalized with that of Gapdh. The relative expression levels (in fold) were determined by using the 2-^{ΔΔct}) method (Livak and Schmittgen, Methods 25, 402-408 (2001)).

### Extracellular Field Potentiation Recording.

Mecp2 cKO mice overexpressed with lenti-mRFP vector, lenti-mRFP-MeCP2WT vector or lenti-mRFP-MeCP2K412R vector in their CA1 area were used for electrophysiological recording. Animals were sacrificed and their brain slices were transferred to an immersion-type recording chamber, perfused with ACSF containing 100 μM picrotoxin at a rate of 2 ml/min at room temperature. An incision was made between the CA1 and CA3 areas to remove afferent input from CA3. For the extracellular field potential recording, a glass pipette filled with 3 M NaCl was positioned in the CA1 stratum radiatum area to record the fEPSP. Bipolar stainless steel stimulating electrodes (Frederick Haer Company, Bowdoin, Me.) were placed in the striatum radiatum to stimulate the Schaffer collateral pathway. Stable baseline fEPSP activity was recorded by applying a short-duration current stimulation pulse (about 40 ps) at a predetermined intensity every 15 s for at least 20 min. LTP was then induced by using both the HFS paradigm and TBS paradigm according to that described previously (Moretti et al., J. Neurosci. 26, 319-327, (2006)). For HFS, LTP was induced by delivering two 100 Hz tetani (1 s) with an inter-tetanus interval of 20 s. For TBS, LTP was induced by delivering three trains of theta-burst stimulation. Each train consisted of 10 sets of bursts (4 stimuli, 100 Hz) with an inter-burst interval of 200 ms. The interval between each stimulus train is 20 s.

### Drugs.

NMDA was purchased from Tocris Bioscience (St. Louis, Mo., USA). IGF-1, corticotropin-releasing factor (CRF) and dexamethasone were purchased from Sigma-Aldrich (St. Louis, Mo.). NMDA, IGF-1 and CRF were dissolved in PBS immediately before use. Dexamethasone was dissolved in DMSO before injection.

### Intra-hippocampal Transfection and Injection.

Rats were anesthetized with pentobarbital (40 mg/kg) and subject to stereotaxic surgery. Two 23-gauge, stainless-steel, thin-wall cannulae were implanted bilaterally to the CA1 area of rat brain at the following coordinates: 3.5 mm posterior to the bregma, ±2.5 mm lateral to the midline and 3.4 mm ventral to the skull surface. After recovery from the surgery, NMDA (2 μg/μl), IGF-1 (100 ng/ml), CRF (100 ng/μl) and dexamethasone (30 ng/μl) were directly injected to the CA1 area at a rate of 0.1 μl/min. A total of 0.7 μl was injected to each side. For transient Mecp2 plasmid DNA transfection, 0.7 μg/μl plasmid DNA complex (1.5 μg/μl) was injected directly to CA1 area bilaterally in the rat brain using the non-viral transfection agent polyethyleneimine (PEI). Before injection, plasmid DNA was diluted in 5% glucose to a stock concentration of 2.77 μg/μl. Branched PEI of 25 kDa (Sigma, St. Louis, Mo.) was diluted to 0.1 M concentration in 5% glucose and added to the DNA solution. Immediately before injection, 0.1 M PEI was added to reach a ratio of PEI nitrogen per DNA phosphate equals to 10. The mixture was subject to vortex for 30 sec and allowed to equilibrate for 15 min. For siRNA injection, 0.7 μl of PIAS1 siRNA (8 pmol) or control siRNA was transfected to CA1 area bilaterally in the rat brain also using the transfection agent PEI. The sense and antisense sequences used for PIAS1 siRNA were adopted from that of a previous study (Tai et al., EMBO J. 30. 205-220 (2011)). The sequence for sense strand was: 5′-UCCGGAUCAUUCUAGAGCUtt-3′ (SEQ ID NO: 18) and that for antisense strand was: 5′-AGCUCUAGAAUGAUCCGGAtt-3′ (SEQ ID NO: 19). The Silencer Negative Control number 1 siRNA was used as a control. They both were synthesized from Ambion (Austin, Tex.). The inner diameter of the injection needle was 0.31 mm and the wall thickness of the injection needle was 0.12 mm. The injection needle was left in place for 5 min to limit the diffusion of injected agent. Animals were sacrificed at different time points after drug injection, plasmid and siRNA transfection. Their brains were removed and cut by a brain slicer. Their CA1 tissue was further punched out by using a stainless punch with 2 mm inner diameter. Tissues were frozen at −80° C. until biochemical experimentation.

### Intra-Basolateral Amygdala Injection of Lentiviral Vectors.

Mice were anesthetized with pentobarbital (40 mg/kg, intraperitoneally) and subject to stereotaxic surgery without cannulation. Lentiviral vectors were directly injected to the BLA after animals recovered from the surgery. The coordinates for the BLA were: 0.5 mm posterior to the bregma, ±3.3 mm lateral to the midline and 4.8 mm ventral to the skull surface. We adopted a replacement assay by co-infected the floxed mice with both a lentivirus encoding Cre and a lentivirus encoding different forms of MeCP2 simultaneously. The recombinase Cre lentivector at a titer of 2×10⁸/ml (diluted in PBS) was mixed with different mRFP-Mecp2 lentivectors also at the titer of 2 ×10⁸/ml (diluted in PBS) for each Mecp2 vector at a 1:1 volume immediate before injection. A volume of 0.25 μl was injected to each side of BLA. The infusion rate was 0.1 μl/min. Social interaction behaviors were measured 7-10 days after lentiviral vector transduction. Mice were sacrificed after the fear conditioning test. Their brains were removed and the BLA tissue was punched out as that described for the CA1 tissue. Tissues were also stored in a −80° C. freezer until further experimentation.

### Three-Chamber Social Ability and Social Novelty Measures.

Social ability and social novelty measures were conducted in a three-chamber cage at the following specifications: 60×40×22 cm (L×W×H). The procedures used for these measures were adopted from that of a previous study (Faizi et al., Brain Behay. 2, 142-154, (2012)). The chamber was divided into three compartments with the left and right compartments of 21 cm in length and the middle compartment of 18 cm in length. There were two additional cylinder chambers with 15 cm in height and 10 cm in diameter placed in both the left and right compartments. During the social ability test, a stranger 1 mouse was placed inside the cylinder in the left compartment with the cylinder on the right compartment empty. The test subject (Mecp2 cKO mouse) was placed in the middle chamber for 10 min and its sniffing time toward stranger 1 and the empty chamber was recorded. At the end of the test, the test subject and stranger 1 were taken out and the chambers were cleaned. Ten minutes later, the test subject and stranger 1 were placed back to their original chambers, respectively. Meanwhile, a stranger 2 mouse was placed in the cylinder on the right compartment. The sniffing time of the test subject to stranger 1 and stranger 2 was recorded during the 10 min observation period and it is regarded as the social novelty test. Social interaction behavior was also measured in rats and the specifications for the three-chamber cage are: 108×50×42 cm (L×W×H).

### Cued Fear Conditioning Learning.

Fear conditioning learning was preformed 7 days after the social interaction test. One day before conditioning, mice were placed in the conditioning chamber (46×30×46 cm, L×W×H) for 5 min for habituation. Twenty-four hours later, these animals were placed into the same chamber for fear conditioning training. After 3 min of free exploration, they were trained with five tone-foot shock pairings. Each tone-shock pairing consisted of a 30-sec tone (85 dB, 10 KHz) which is co-terminated with a foot shock (0.1 mA, 1 sec). After shock presentation, a 60-sec intertrial interval preceded the next tone and foot shock. Twenty-four hours later, these animals were placed into another chamber for the retention test. After 3 min of free exploration, a 30-sec tone (85 dB, 10 KHz) was presented without foot shock. Five tones were given in the retention test with 60-sec intervals. The freezing response of mice was calculated as the percentage of time spent freezing during the 30-sec tone period. The parameters used for fear conditioning learning were adopted from that of a previous study (King et al., Learn Mem. 16, 625-634 (2009)).

### Immunohistochemistry.

For immunohistochemical staining of PIAS1 and MeCP2 in CA1 area of the rat brain, rats were anesthetized with pentobarbital (100 mg/kg, i.p.) and perfused with ice-cold phosphate-buffered saline followed by 4% paraformaldehyde. Brains were removed and post-fixed in 20% sucrose/4% paraformaldehyde solution for 20-48 h. Brains were then frozen, cut into 30-μm sections on a cryostat and mounted on gelatin-coated slides. Brain sections were rinsed with 1×PBS for 10 min and permeabilized with pre-cold EtOH/CH3COOH (95%:5%) for 10 min followed by 1×PBS for 10 min for three times. The sections were pre-incubated in a blocking solution containing 3% normal goat serum, 3% BSA, and 0.2% Triton X-100 in 1×PBS for 2 h followed by 1×PBS for 10 min for three times. For visualization of endogenous PIAS1 and MeCP2 in hippocampal CA1 area, brain sections were incubated with rabbit anti-PIAS1 antibody (1:100, Catalog No. 2474-1, Epitomics) and mouse anti-MeCP2 antibody (1:100, Catalog No. H00004204-M01, Abnova, Taipei, Taiwan) at 4° C. overnight. Brain sections were then washed with 1 ×PBS for 10 min for three times and then incubated with goat anti-rabbit secondary antibody conjugated with FITC (1:500, Catalog No. 111-095-003, Jackson Immunoresearch, West Grove, Pa.) and Cy3 donkey anti-mouse antibody (1:500, Catalog No. GTX85338, Genetex) for 1 h. For immunofluorescence detection of the nucleus, tissue sections were added with 20 μl of the VECTASHIELD mounting medium with DAPI (1.5 μg/ml) (Vector Laboratories, Burlingame, Calif.). For examination of lentiviral vector transduction in BLA, brain sections were prepared for visualization of GFP (green) and RFP (red) fluorescence. Photomicrographs were taken using a Zeiss LSM510 confocal microscope.

### Primary Hippocampal Culture and Immunocytochemistry.

Embryonic primary hippocampal neurons were prepared from E18 of Sprague-Dawley rats. The hippocampus from the embryo was dissociated with 100 U/ml papain and plated onto 100 μg/ml poly-L-lysine coated coverslips at a density of 3×10⁵ cells/ml with minimal essential medium containing 5% fetal bovine serum and 5% horse serum. Three hours later, the medium was replaced with neurobasal medium (Catalog No. 21103049, Invitrogen) containing B27 supplements (Catalog No. 17504044, Invitrogen), GlutaMAX supplement (Catalog No. 35050061, Invitrogen), 100 units/ml Penicillin and 100 μg/ml Streptomycin (Catalog No. 15140122, Invitrogen). Cultures were maintained at 37° C. in a humidified atmosphere at 5% CO₂ and the neurobasal medium was replaced again two days later. For immunofluorescence visualization of PIAS1 and MeCP2 in dissociated neurons, cultured hippocampal neurons at DIVS were fixed with 4% paraformaldehyde-4% sucrose (wt/vol) at room temperature for 10 min, the paraformaldehyde/sucrose mixture was then aspirated and 1 ml of 0.25% (vol/vol) Triton X-100 in PBS was added to each well at room temperature for 10 min. The coverslips were washed carefully with PBS and added with 1 ml of 5% (wt/vol) bovine serum albumin (BSA) in PBS at room temperature for 1 h. The primary antibodies, rabbit anti-PIAS1 antibody (1:300, Catalog No. 2474-1, Epitomics, Burlingame, Calif.) and mouse anti-MeCP2 antibody (1:300, Catalog No. 61285, Active Motif), were diluted in 1% (wt/vol) BSA in PBS, added to the coverslip and incubated at 4° C. for overnight. The coverslips were then washed three times gently with PBS. After PBS washing, DyLight 488 goat anti-rabbit antibody (1:1000, Catalog No. GTX76757, Genetex) and Cy3 donkey anti-mouse antibody (1:1000, Catalog No. GTX85338, Genetex) were diluted in 1% (wt/vol) BSA in PBS and added to the coverslips for 1 h. The coverslips were then washed three times with PBS and added with 20 μl VECTASHIELD mounting medium with DAPI (Vector Laboratories). Photomicrographs were taken using a Zeiss LSM510 confocal microscope.

### Statistics.

Behavioral data were analyzed with one-way or two-way analysis of variance (ANOVA) with repeated measure followed by post-hoc Newman-Keuls multiple comparisons (represented by q value). Biochemical data were analyzed with the Student's t-test or one-way ANOVA followed by Newman-Keuls comparisons. Electrophysiological data were analyzed with two-way ANOVA with repeated measure followed by Newman-Keuls comparisons. Values of P<0.05 were considered statistically significant (*P<0.05, **P<0.01, #P<0.001).

### Example 1

### Identifying Candidate SUMO Sites in MeCP2

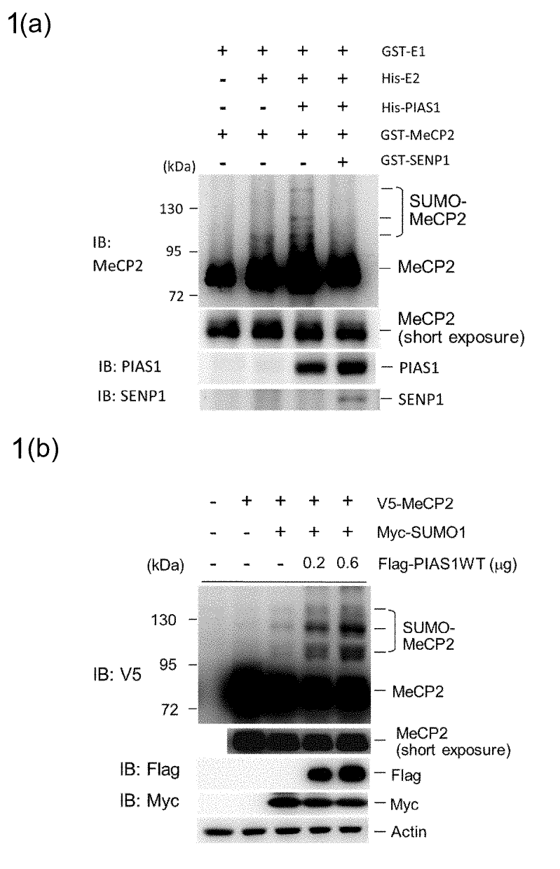

In this example, the candidate SUMOlyation sites of MeCP2 were investigated uisng in vitro SUMOylation assays. Results are illustrated in FIG. 1a. Data in FIG. 1 a showed that MeCP2 SUMOylation occurred when E1, E2, PIAS1 (the protein inhibitor of activated STAT1) and MeCP2 proteins were added, but such effect was blocked by the addition of sentrin-specific protease 1 (SENP1), an enzyme that removes the sumo molecule from sumo-conjugated protein.

Next, the possibility of MeCP2 being sumoylated by PIAS1 in the cells was investigated. To this purpose, HEK293T cells were first transfected with V5-MeCP2, Myc-SUMO1 and Flag-PIAS1 (in different amount) plasmids, respectively; followed by in vitro SUMOylation assay. Results are depicted in FIG. 1b. It was found that PIAS1 enhanced the SUMOylation of MeCP2 in a dose-dependent manner.

Then, the candidate SUMO acceptors on MeCP2 were investigated using mass spectrometric (MS). The MS result revealed 10 candidate SUMO residues on MeCP2, but none of them fits the consensus SUMO-substrate motif. Thus, the bioinformatics method and SUMO2.0 Software were used to try identifying the candidate residues. Two lysine residues exhibited higher scores, and one of them (Lys-363) fits to the consensus SUMO-substrate motif. In addition, based on SUMO2.0 Software prediction of candidate SUMO acceptors on MeCP2, four additional lysine residues were found to exhibit medium scores (Table 1).

TABLE 1
SUMO2.0 Software prediction of candidate SUMO acceptors on MeCP2.
The “K” letter indicated by the arrow represents the candidate SUMO sites.

| Position | peptide | Type |
|---|---|---|
| High score | ↓ | |
| 32 | KKVKKDK | Non-consensus |
| 363 | SPPKKEH | ψ-K-X-E |
| Medium score | | |
| 12 | REEKSED | Non-consensus |
| 35 | KKDKKED | Non-consensus |
| 36 | KDKKEDK | Non-consensus |
| 412 | SICKEEK | Non-consensus |

Accordingly, individual mutants against these six residues were generated, and each mutant (V5-tagged), together with Flag-PIAS1 and Myc-SUMO1, were used to transfect HEK293T cells. Results are depicted in FIG. 1c. SUMOylation of MeCP2 occurred when the cells were transfected with V5-MeCP2WT, but the effect was blocked when cells were transfected with Myc-SUMO1ΔGG, the SUMO1 plasmid that lacked the C-terminal di-glycine motif essential for SUMO1 conjugation (FIG. 1c). Transfecting the cells with V5-MeCP2K12R, V5-MeCP2K32R, V5-MeCP2K35R and V5-MeCP2K36R did not alter MeCP2 SUMOylation. MeCP2 SUMOylation diminished when cells were transfected with V5-MeCP2K363R, in which the upper MeCP2 sumo-band was blocked (FIG. 1c). Further, expression of the upper and middle MeCP2 sumo-bands were suppressed when cells were transfected with V5-MeCP2K412R (FIG. 1c). The overall SUMOylation intensity was significantly reduced by V5-MeCP2K412R transfection than by V5-MeCP2K363R transfection (FIG. 1c).

Further analysis confirmed that MeCP2 SUMOylation occurred at Lys-412 in the cell. Flag-PIAS1, Myc-SUMO1, V5-MeCP2WT or V5-MeCP2K412R plasmids were used to transfect HEK293T cells. The cells were immunoprecipitated with anti-Myc antibody and immunoblotted with anti-V5 antibody. Results in FIG. 1e depicted that the SUMOylation intensity was decreased by V5-MeCP2K412R transfection (FIG. 1d).

Since MeCP2K412R possessed the most significant effect in decreasing MeCP2 SUMOylation than MeCP2K363R did, subsequent experiments focused on Lys-412 only. In addition, one previous report indicated that MeCP2 could be SUMO-modified at Lys-223 (Cheng et al., J. Neurochem. 128, 798-806 (2014)), MeCP2 SUMOylation at this residue was also monitored. V5-MeCP2WT or V5-MeCP2K223R was co-transfected with Flag-PIAS1 and Myc-SUMO1 to HEK293T cells and MeCP2 SUMOylation was examined. V5-MeCP2K412R was transfected as a positive control. Result revealed that MeCP2 SUMOylation was not altered by V5-MeCP2K223R transfection (data not shown).

### Example 2

### MeCP2 is SUMO-Modified By PIAS1 at Lys-412 in the Hippocampus

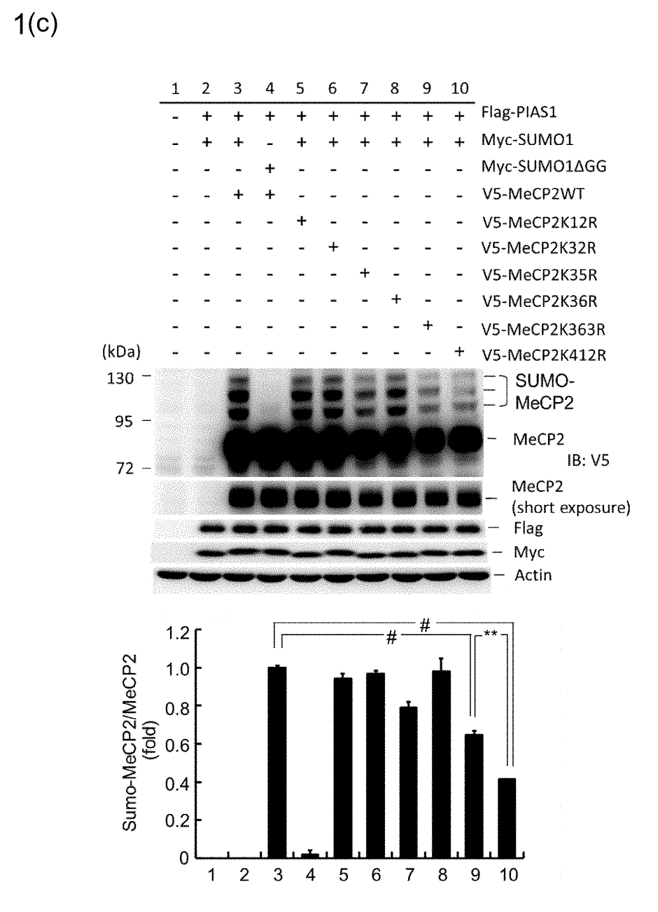

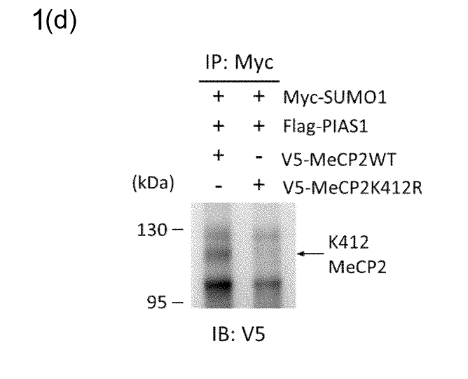

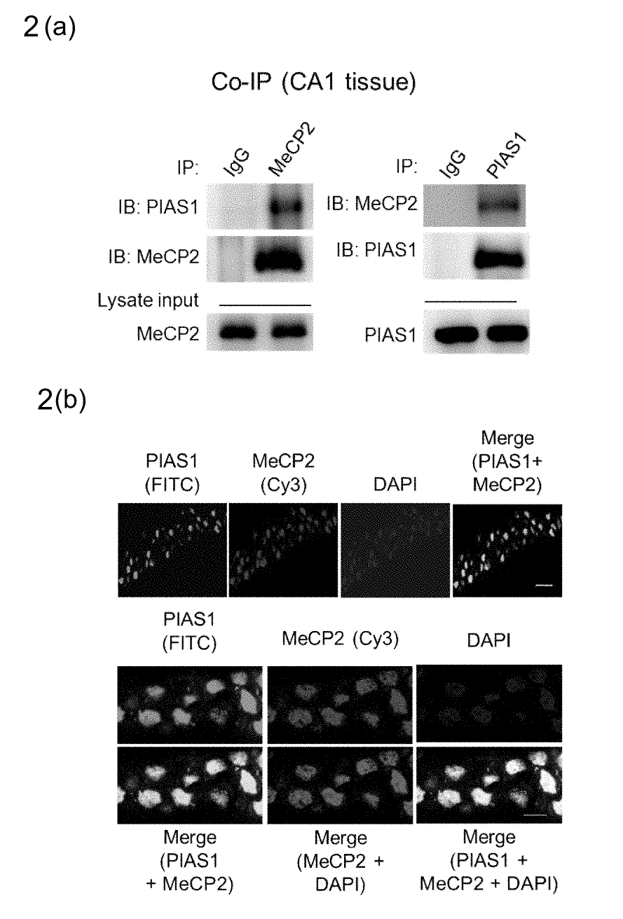

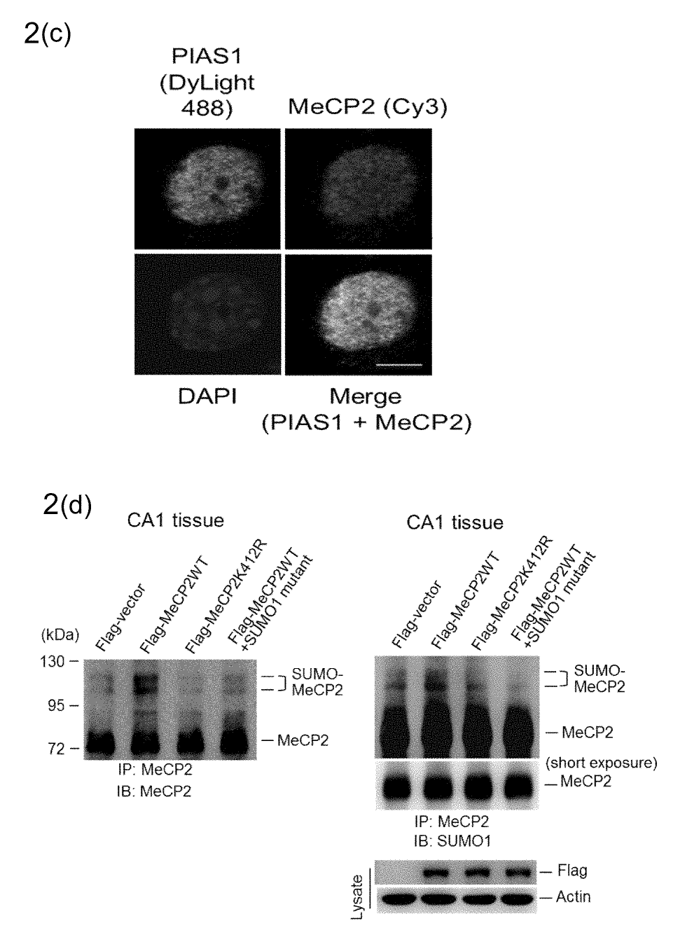

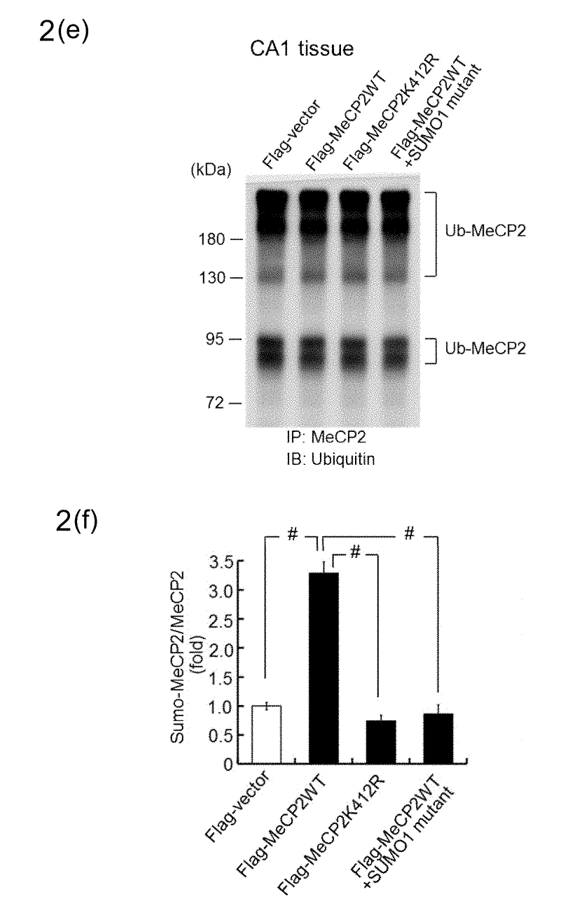

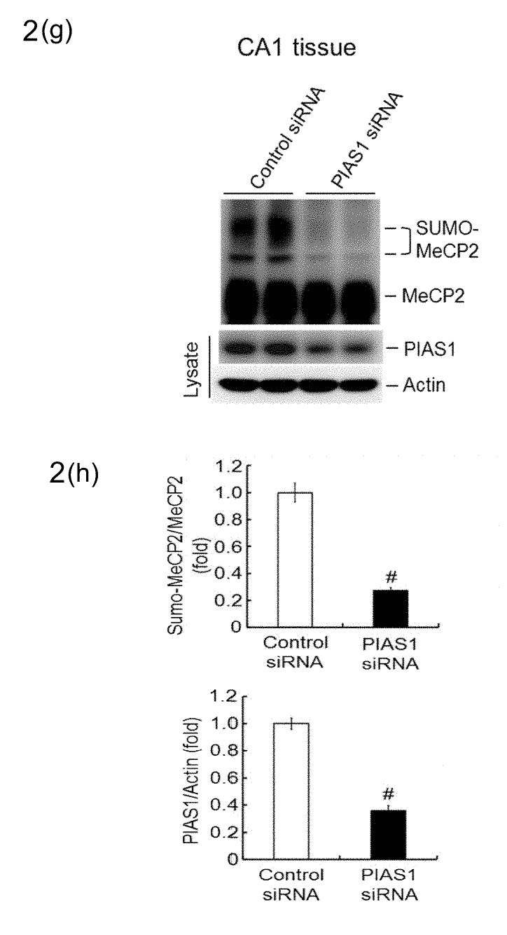

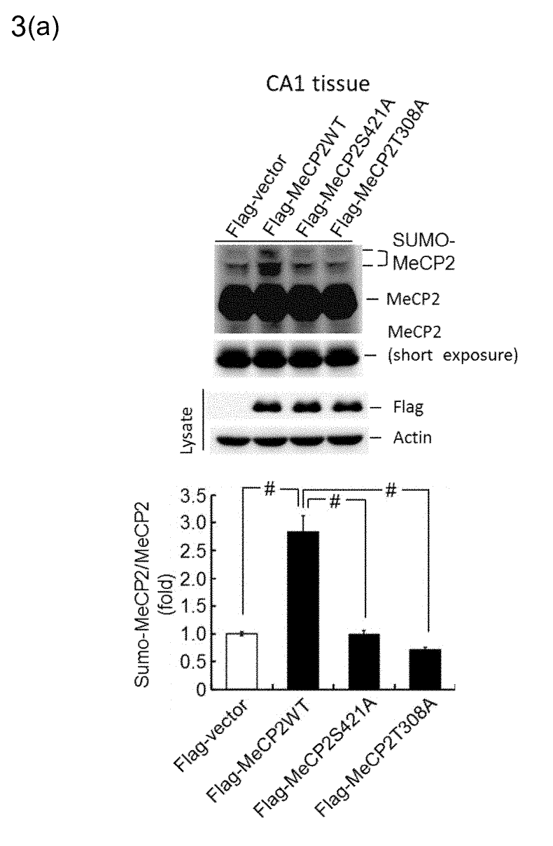

The results in Example 1 indicated that MeCP2 may be sumoylated by PIAS1 at Lys-412 in the cell, in this example, whether MeCP2 may be SUMO-modified by PIAS1 in the hippocampus of rat brain was investigated. Co-immunoprecipitation (co-IP) was first carried out. Results are illustrated in FIG. 2a, which indicated that when cells were immunoprecipitated with anti-MeCP2 antibody and immunoblotted with anti-PIAS1 antibody, MeCP2 was apparently associated with PIAS1 (FIG. 2a, left panel). Similar results were obtained when cells were immunoprecipitated with anti-PIAS1 antibody and immunoblotted with anti-MeCP2 antibody (FIG. 2a, right panel).

Next, whether PIAS1 and MeCP2 are present in the same hippocampal neurons were investigated. Brain sections containing the CA1 region were subject to immunohistochemistry staining. Results are depicted in FIG. 2b. The immunofluorescence for PIAS1 (green), MeCP2 (red) and DAPI (blue) were respectively visualized in CA1 neurons (FIG. 2b, upper panel). When CA1 neurons were visualized at a higher magnification, both PIAS1 and MeCP2 were found present and co-localized in the nucleus of the same hippocampal neurons (FIG. 2b, lower panel). For better visualization of PIAS1 and MeCP2 distribution in individual neurons, cultured hippocampal neurons were subject to immunocytochemistry staining of PIAS1 and MeCP2. Results revealed that both PIAS1 and MeCP2 were present and co-localized in the nucleus of the same hippocampal neuron, and puncta distribution of MeCP2 was observed at heterochromatins (FIG. 2c).

Next, whether MeCP2 may be sumoylated at Lys-412 in the hippocampus was investigated. Flag-vector, Flag-MeCP2WT or Flag-MeCP2K412R was transfected to rat CA1 area and in vitro SUMOylation assay was performed 48 hrs later. Results indicated that transfection of Flag-MeCP2WT significantly increased the level of MeCP2 SUMOylation, but this effect was blocked by Flag-MeCP2K412R transfection. Addition of the SUMO1 mutant protein also blocked the enhancing effect of Flag-MeCP2WT on MeCP2 SUMOylation (compared with the Flag-MeCP2WT group) (FIG. 2d, left panel). These results were confirmed when cells were immunoprecipitated with anti-MeCP2 antibody and immunoblotted with anti-SUMO1 antibody (FIG. 2d, right panel). Plasmid transfection and expression was confirmed by western blot using anti-Flag antibody (FIG. 2d, lower-right panel). Since SUMOylation and ubiquitination both occurred at lysine residues, to rule out the possibility that the observed SUMO-MeCP2 bands were in fact ubiquitinated MeCP2, the same cell lysates were immunoprecipitated with anti-MeCP2 antibody but immunoblotted with anti-ubiquitin antibody. Results were summarized in FIGS. 2d and 2e, which indicated that the molecular weight of ubiquitinated MeCP2 bands was either below 95 kDa or above 130 kDa (FIG. 2e), differed from that of the sumoylated MeCP2 bands, which were located between 95 kDa and 130 kDa (FIG. 2d). The quantified result of MeCP2 SUMOylation was depicted in FIG. 2f. Plasmid transfection and expression in CA1 neurons was further confirmed by immunohistochemistry using anti-Flag antibody and FITC-conjugated secondary antibody (data not shown).

We next addressed the issue whether MeCP2 is sumoylated by PIAS1 endogenously. Rats received control siRNA or PIAS1 siRNA (8 pmol) transfection to their CA1 area, and endogenous MeCP2 SUMOyation was determined by in vitro

SUMOylation assay 48 hrs later. Results revealed that knockdown PIAS1 would significantly decrease the level of endogenous MeCP2 SUMOylation (FIG. 2g). The quantified result is shown in FIG. 2h (upper panel). The effectiveness of PIAS1 siRNA transfection was confirmed by decreased PIAS1 expression in CA1 area (FIG. 2h, lower panel).

### Example 3

### MeCP2 Phosphorylation Facilitates MeCP2 SUMOylation and MeCP2 SUMOylation is Induced by NMDA and IGF-1 and by CRF Treatments

### 3.1 MeCP2 Phosphorylation Facilitates MeCP2 SUMOylation

Previous studies indicated that MeCP2 phosphorylation at Ser-421 and Thr-308 is induced by neuronal activation (Zhou et al., Neuron 52, 255-269 (2006); Ebert et al., Nature 499, 341-345 (2013)). In this example, whether MeCP2 phosphorylation may facilitate MeCP2 SUMOylation was investigated.

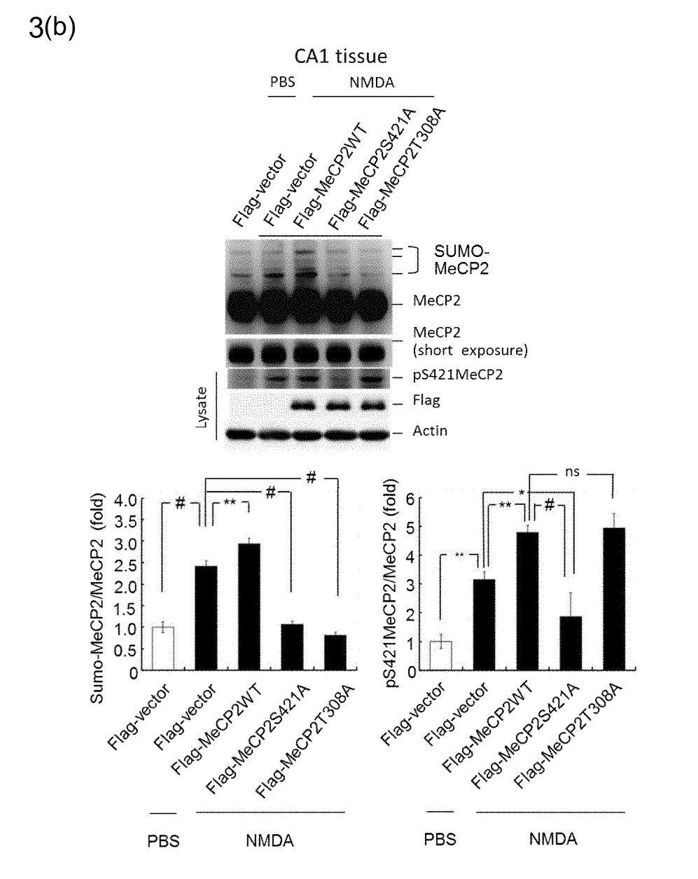

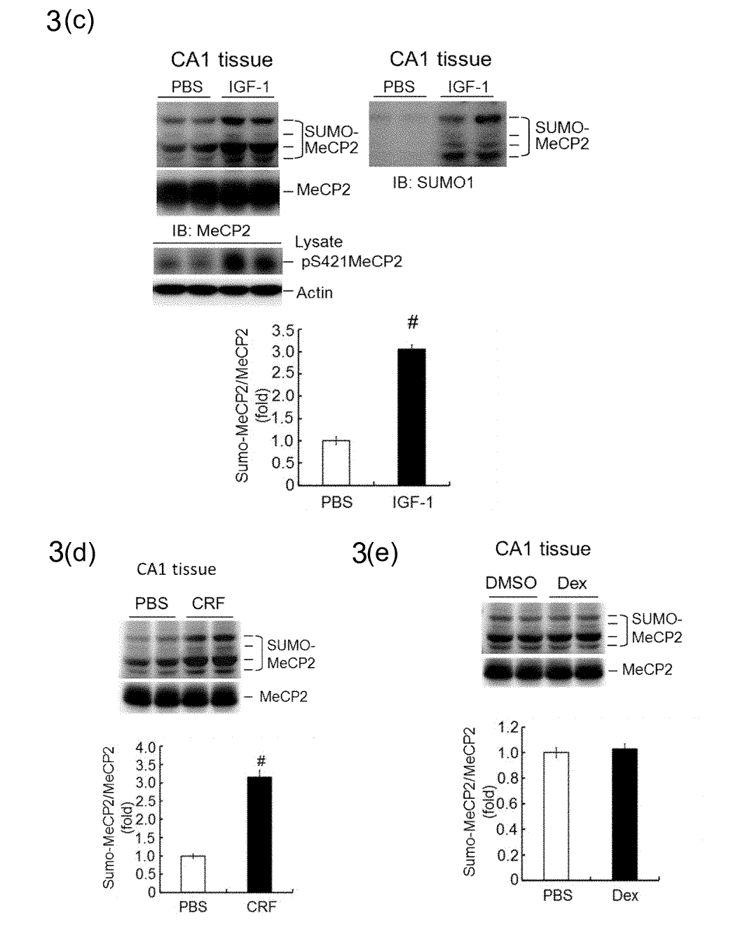

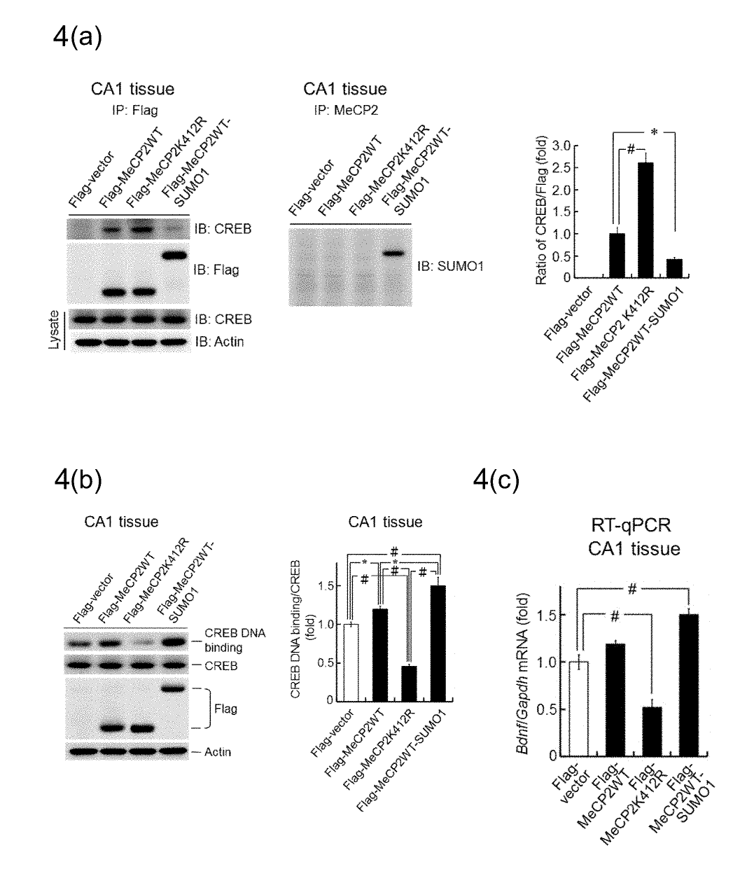

Rats received Flag-vector, Flag-MeCP2WT, Flag-MeCP2S421A or Flag-MeCP2T308A transfection to their CA1 area and in vitro SUMOylation assay was performed 48 hrs later. Results indicated that the level of MeCP2 SUMOylation increased when rats received Flag-MeCP2WT transfection, but this effect was blocked by either Flag-MeCP2S421A or Flag-MeCP2T308A transfection (FIG. 3a).

### 3.2 MeCP2 SUMOylation is Induced by NMDA and IGF-1 and CRF

Whether MeCP2 SUMOylation may be induced by neuronal activation using N-methyl-D-aspartate (NMDA) as a stimulus and whether the two phosphorylation mutants block NMDA-induced MeCP2 SUMOylation were investigated. To this purpose, rats received Flag-vector, Flag-MeCP2WT, Flag-MeCP2S421A or Flag-MeCP2T308A transfection as described above. NMDA injection (2 μg/μl) was made to the CA1 area of these rats 47 hrs after plasmid transfection. The animals were sacrificed 1 hr after NMDA injection and their CA1 tissues were respectively punched out for in vitro SUMOylation assay. Another group of rats receiving Flag-vector transfection and PBS injection served as the control. Results revealed that NMDA injection increased the level of MeCP2 SUMOylation. This effect was further enhanced by Flag-MeCP2WT transfection, but was abolished by Flag-MeCP2S421A and Flag-MeCP2T308A transfections (FIG. 3b). Consistent with previous reports that neuronal activation increased the phosphorylation levels of MeCP2 at Ser-421 and Thr-308 (Zhou et al., Neuron 52, 255-269 (2006); Ebert et al., Nature 499, 341-345 (2013), NMDA injection also increased the phosphorylation level of MeCP2 at Ser-421 (FIG. 3b). This effect was similarly enhanced by Flag-MeCP2WT transfection, but was blocked by Flag-MeCP2S421A transfection. It was un-affected by Flag-MeCP2T308A transfection (FIG. 3b). The quantified results are shown in the lower panel.

Insulin-like growth factor-1 (IGF-1) has been suggested as a potential treatment for RTT patients (Pini et al., Autism Res Treat 2012, 679801 (2012); Pine et al., Front Pediatr. 2, 52 (2014)), thus whether MeCP2 SUMOylation may be a molecular target of IGF-1 was investigated. Rats received PBS or IGF-1 (100 ng/ml) injection to their CA1 area and MeCP2 SUMOylation was determined 1 hr later. Results revealed that IGF-1 significantly increased the level of MeCP2 SUMOylation. In addition, IGF-1 seemed to yield more sumo-bands and this was evidenced by immunoblotting with both anti-MeCP2 (FIG. 3c, left panel) and anti-SUMO1 (FIG. 3c, right panel) antibodies. Meanwhile, IGF-1 increased the phosphorylation level of MeCP2 at Ser-421 (FIG. 3c, lower-left panel).

Corticotropin-releasing factor (CRF) and dexamethasone (a synthetic glucocorticoid) are also known to enhance cognitive function and Bdnf gene expression, and may have the potential to ameliorate the RTT syndrome. Thus, the effect of CRF or dexamethasone on MeCP2 SUMOylation was investigated. To this purpose, rats received PBS or CRF (100 ng/pl) injection to their CA1 area, and were sacrificed 1 hr later and their CA1 tissues were subject to in vitro SUMOylation assay. In another experiment, rats received DMSO or dexamethasone (30 ng/pl) injection to their CA1 area, and were also sacrificed 1 hr later and their CA1 tissues were subject to MeCP2 SUMOylation determination. Results revealed that CRF markedly increased the level of MeCP2 SUMOylation (FIG. 3d), however, dexamethasone did not produce the same effect (FIG. 3e).

### Example 4

### SUMOylation of MeCP2 Decreases its Interaction with CREB and Increases CREB DNA Binding and Bdnf Gene Expression and Methyl-DNA Binding

In this experiment, the molecular events downstream of MeCP2 SUMOylation were investigated.

4.1 SUMOylation of MeCP2 Decreases its Interaction with CREB

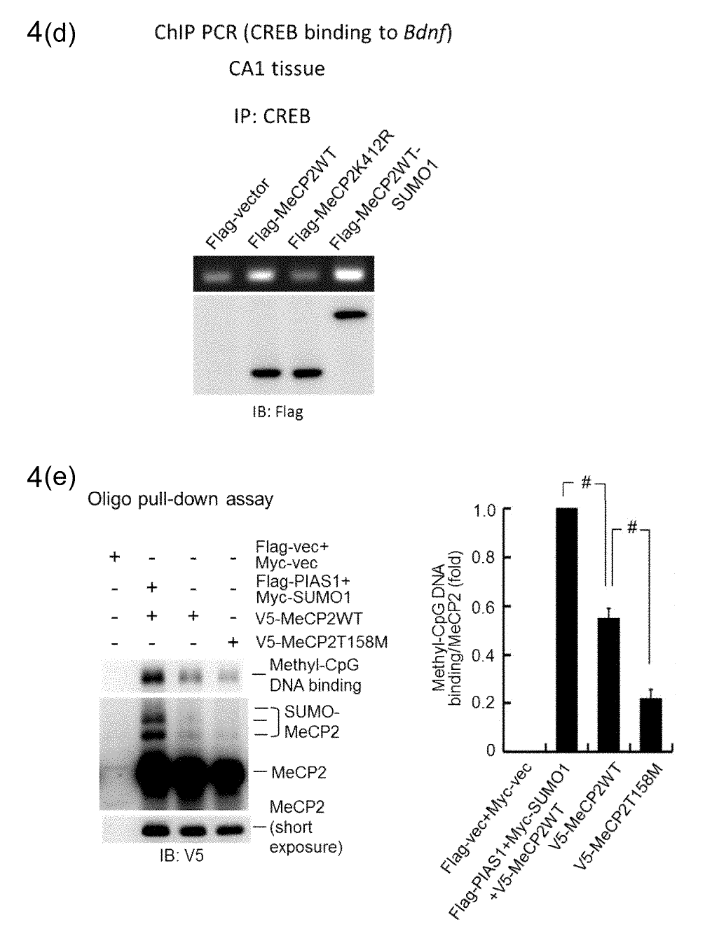

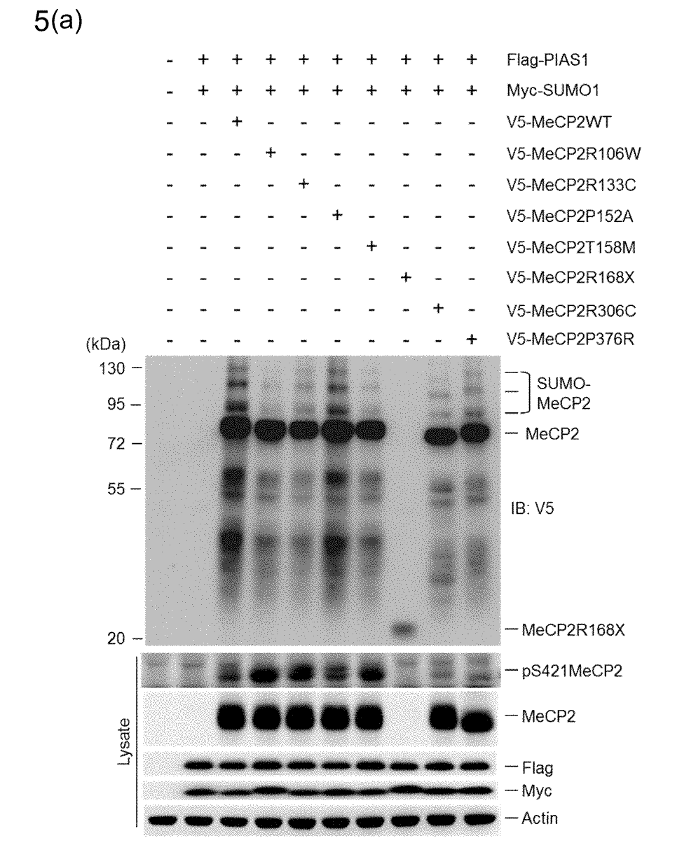

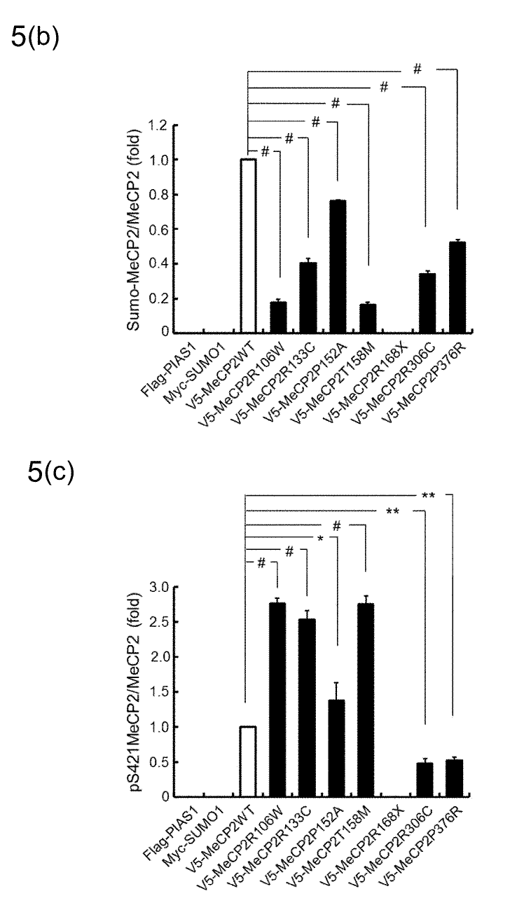

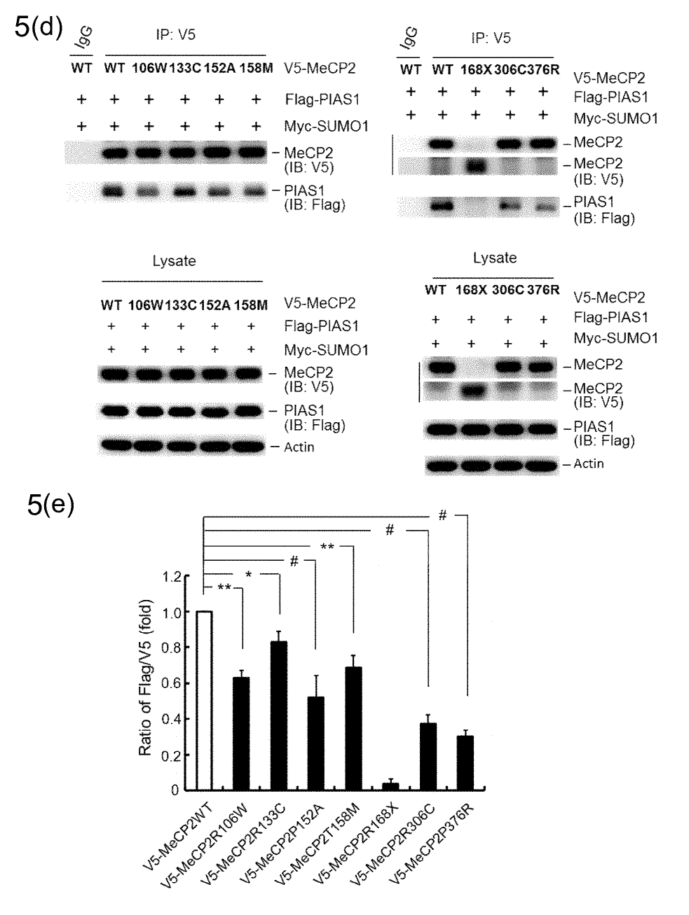

Since Bdnf plays an important role in RTT and CREB binds to the Bdnf promoter directly, thus, whether SUMOylation of MeCP2 altered its interaction with CREB was first investigated. Rats received Flag-vector, Flag-MeCP2WT, Flag-MeCP2K412R or Flag-MeCP2WT-SUMO1 transfection to their CA1 area. They were sacrificed 48 hrs later and their CA1 tissues were subject to co-IP experiment. Results revealed that MeCP2 was associated with CREB; this association was increased by Flag-MeCP2K412R transfection and diminished by Flag-MeCP2WT-SUMO1 transfection (FIG. 4a, left panel). To further confirm that the higher molecular weight band indicated sumoylated MeCP2, cell lysates from the same groups were immunoprecipitated with anti-MeCP2 antibody and immunoblotted with anti-SUMO1 antibody. Result revealed that a specific band was observed at the same molecular weight in the Flag-MeCP2-SUMO1 group only (FIG. 4a, middle panel). The quantified result is shown in the right panel.

### 4.2 MeCP2 SUMOylation Increases CREB DNA Binding

One possible explanation for the above result is that upon MeCP2 SUMOylation, CREB is released from the MeCP2 repressor complex and becomes more available for DNA binding and transcriptional regulation. This hypothesis was thus examined here. Flag-vector, Flag-MeCP2WT, Flag-MeCP2K412R and Flag-MeCP2WT-SUMO1 were transfected to rat CA1 area. Animals were sacrificed 48 hrs later and their CA1 tissues were subject to CREB DNA binding determination. Results revealed that Flag-MeCP2WT increased, whereas Flag-MeCP2K412R decreased CREB DNA binding, when compared with that of the control group. Transfection of Flag-MeCP2WT-SUMO1 further increased CREB DNA binding, as compared with that of the Flag-MeCP2WT group (FIG. 4b, left panel). The expression level of CREB was not altered by these plasmid transfections.

### 4.3 MeCP2 SUMOylation Increases Bdnf Gene Expression

Since blockade of MeCP2 SUMOylation decreases CREB DNA binding and CREB directly binds to the Bdnf promoter, we then examined whether SUMOylation of MeCP2 may regulate Bdnf promoter activity. Different V5-tagged MeCP2 plasmids were transfected to Neuro2A cells with co-transfection of the Bdnf exon IV promoter construct and the Renilla luciferase-encoding construct (as an internal control). Bdnf promoter activity was determined 48 hrs later by reporter luciferase assay. Results indicated that V5-MeCP2K412R decreased, whereas V5-MeCP2WT-SUMO1 increased Bdnf promoter activity (data not shown). We further examined the transfection efficiency in this experiment. mRFP-MeCP2WT was transfected to Neuro2A cells; total number of cells and the number of cells that showed red fluorescence were counted 48 hrs later. Results revealed that the transfection efficiency is approximately 40% (data not shown).

The issue of whether MeCP2 SUMOylation would regulate Bdnf mRNA expression was also investiged. To this purpose, rats received Flag-vector, Flag-MeCP2WT, Flag-MeCP2K412R or Flag-MeCP2WT-SUMO1 transfections. They were sacrificed 48 hrs later and their CA1 tissues were subject to Bdnf mRNA determination. Results indicated that transfection of Flag-MeCP2WT increased Bdnf mRNA level for approximately 18%, but transfection of Flag-MeCP2K412R decreased Bdnf mRNA level for about 50%, as compared with that of the control group. Further, transfection of Flag-MeCP2WT-SUMO1 increased Bdnf mRNA level for about 50% (FIG.

4c). Because MeCP2 may regulate the expression of a wide variety of genes, a cDNA microarray analysis was performed in this regard. Results revealed that Flag-MeCP2K412R transfection altered the expression of approximately 40,000 genes, as compared with that of Flag-MeCP2WT transfection. Among these genes, 1,368 genes exhibited 2 fold up-regulation, and 1,686 genes exhibited 2 fold down-regulation (data not shown). To validate the result obtained from microarray analysis, three genes that were up-regulated by Flag-MeCP2K412R transfection and three genes that were down-regulated by Flag-MeCP2K412R transfection were respectively selected for further RT-qPCR analysis. Results revealed that Flag-MeCP2K412R increased the mRNA level of Olr640, Bco2 and Mx1 genes for approximately 9 fold, 4.3 fold and 3.8 fold, respectively; and it decreased the mRNA level of Igf2, Wnt6 and Wnt5b genes for approximately 5 fold, 3.6 fold and 1.7 fold, respectively (data not shown). Although the fold differences were not the same as that obtained from microarray analysis, the RT-qPCR results are in general consistent with the results obtained from microarray analysis. However, the Bdnf gene was not on the list of microarray analysis. It was not known whether the time point we selected for microarray analysis (48 hrs) is not best for detection of Bdnf mRNA alteration. Therefore, we have conducted a time-course study to include the time intervals of 24 hrs, 36 hrs and 48 hrs to further examine the effect of Flag-MeCP2K412R transfection on Bdnf mRNA expression in separate groups of animals. Results revealed that Flag-MeCP2K412R transfection significantly decreased Bdnf mRNA level at all time intervals examined (data not shown).

The issue whether MeCP2 SUMOylation would increase CREB binding to endogenous Bdnf promoter in the hippocampus was also examined. Flag-vector, Flag-MeCP2WT, Flag-MeCP2K412R and Flag-MeCP2WT-SUMO1 were transfected to rat CA1 area. Animals were sacrificed 48 hrs later and their CA1 tissues were subject to chromatin immunoprecipitation (ChIP) assay. Results revealed that CREB directly bound to the endogenous Bdnf promoter. Blockade of MeCP2 SUMOylation by Flag-MeCP2K412R transfection decreased, whereas enhanced MeCP2 SUMOylation by Flag-MeCP2WT-SUMO1 transfection increased CREB binding to the Bdnf promoter, as compared to that of the Flag-MeCP2WT group (FIG. 4d).

4.4 MeCP2 SUMOylation increases its methyl-DNA binding.

Several MECP2 mutations identified in RTT patients exhibited either altered interaction with other proteins or impaired methyl-DNA binding (Ballestar, Biochemistry 39, 7100-7106 (2000)), the issue of whether MeCP2 SUMOylation alters its methyl-DNA binding was thus investigated. V5-MeCP2WT was transfected to HEK293T cells with or without the co-transfection of Flag-PIAS1 and Myc-SUMO1. V5-MeCP2T158M was transfected as a negative control. MeCP2 methyl-DNA binding was observed when Flag-MeCP2WT was transfected, however, co-transfection of Flag-PIAS1 and Myc-SUMO1 markedly increased MeCP2 methyl-DNA binding; it also increased MeCP2 SUMOylation (FIG. 4e). Whereas transfection of V5-MeCP2T158M almost completely abrogated MeCP2 methyl-DNA binding and MeCP2 SUMOylation (FIG. 4e).

### Example 5

### Several MECP2 Mutations Identified in RTT Patients Exhibit Decreased Level of MeCP2 SUMOylation and Decreased Interaction with PIAS1

In this example, whether MECP2 mutations identified in RTT patients exhibit abnormal MeCP2 SUMOylation and/or interaction towards PIAS1 were investigated.

5.1 MECP2 mutations seen in RTT exhibited decreased MeCP2 SUMOylation

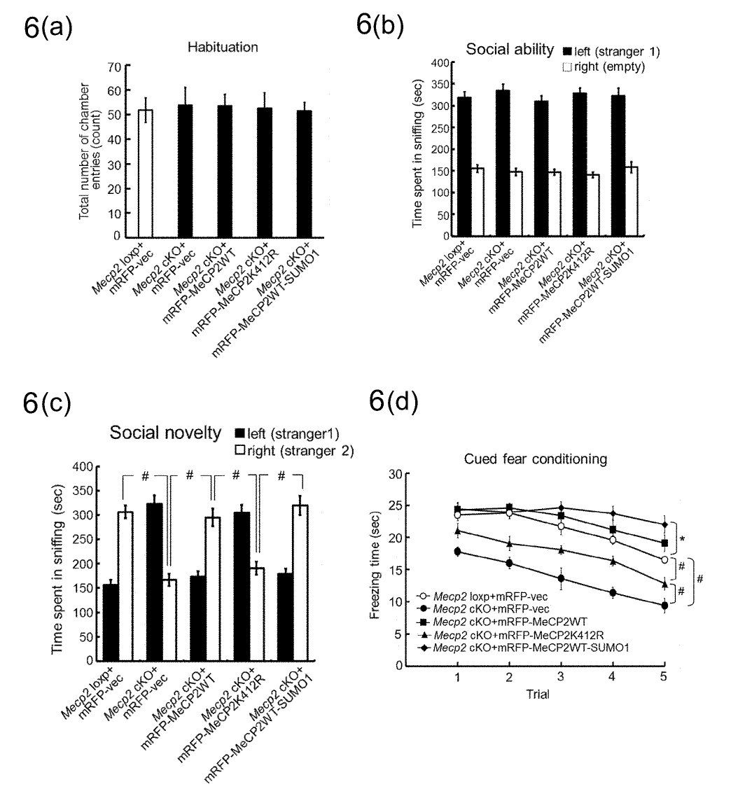

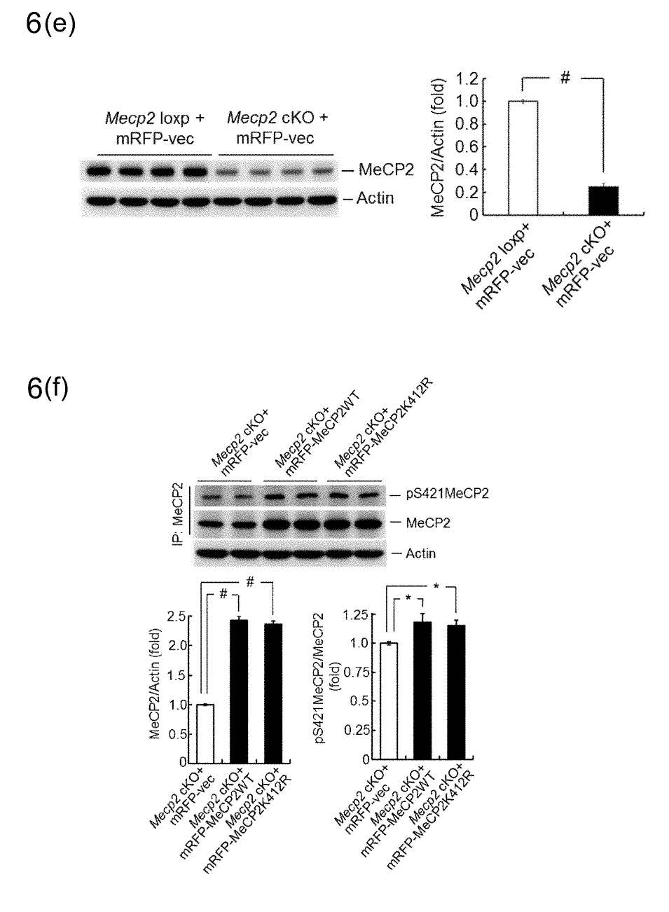

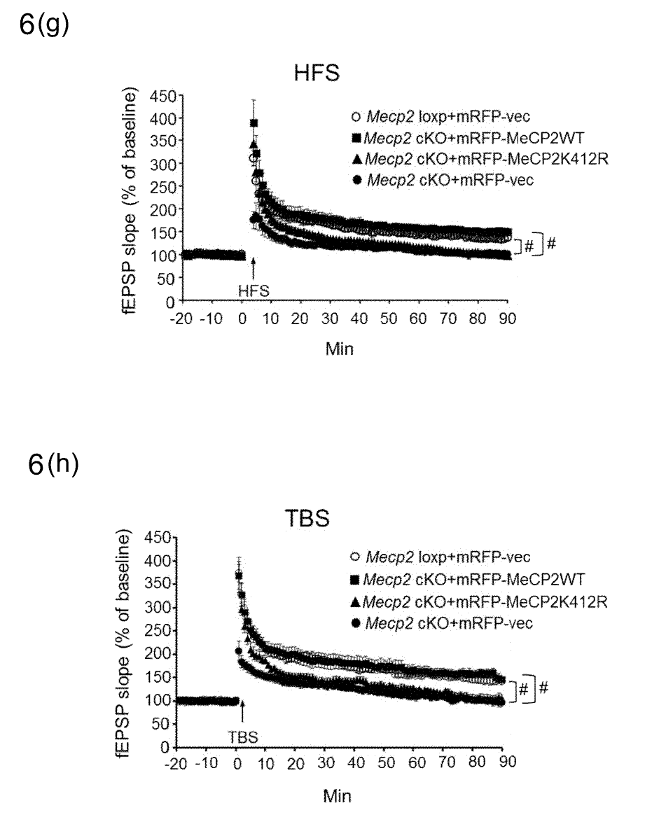

The level of MeCP2 SUMOylation in seven MECP2 mutations that are most commonly seen in RTT patients were investigated. Individual V5-tagged MECP2 mutant plasmid was transfected to HEK293T cells and in vitro SUMOylation assay was performed 48 hrs later. Results revealed that MECP2 mutants R106W, R133C, P152A, T158M, R306C and P376R all significantly decreased MeCP2 SUMOylation with different extent. R168X mutant did not exhibit any MeCP2 SUMOylation because it was a truncated protein (FIG. 5a). The quantified result is illustrated in FIG. 5b. However, these mutations do not cover the residues that are sumoylated (Lys-363, Lys-412) or phosphorylated (Thr-308, Ser-421) that may regulate MeCP2 SUMOylation. To investigate why these mutants exhibited decreased SUMOylation levels, we examined whether they altered the level of MeCP2 phosphorylation. Results indicated that only MeCP2R306C and MeCP2P376R exhibited decreased MeCP2 phosphorylation; other MeCP2 mutants exhibited increased MeCP2 phosphorylation (FIG. 5a). The quantified result is depicted in FIG. 5c.

5.2 MECP2 mutations of RTT show decreased interaction with PIAS1

The cause of reduced MeCP2 SUMOylation seen in above mutants was investigated by determining if there were decreased interaction between PIAS1 and these MECP2 mutants. Individual VS-tagged MeCP2 mutant plasmid was co-transfected with Flag-PIAS1 and Myc-SUMO1 to HEK293T cells and co-IP was carried out 48 hrs later. Results revealed that all the MeCP2 mutants exhibited reduced association with PIAS1 (FIG. 5d, upper panels). The quantified result is depicted in FIG. 5e. The expression level of these transfected plasmids in cell lysates was similar (FIG. 5d, lower panels). The association between PIAS1 and MeCP2K223R was also examined. Result revealed that the interaction between PIAS1 and MeCP2K223R was not altered, as compared with that of the MeCP2WT group (data not shown).

### Example 6

### MeCP2 SUMOylation Rescues Mecp2 cKO Mice-Induced Behavioral and LTP Deficits

The functional significance of MeCP2 SUMOylation was investigated in this example by use of Mecp2 cKO mice.

To generate Mecp2 knockout animals, mice were respectively treated with lenti-mRFP-vector, lenti-mRFP-MeCP2WT vector, lenti-mRFP-MeCP2K412R vector and lenti-mRFP-MeCP2WT-SUMO1 fusion vector in the basolateral amygdala (BLA), and were subsequently subject to habituation and social interaction measures. The Mecp2 loxp mice receiving lenti-mRFP-vector transduction served as the control. Anatomical localization of GFP-Cre expression and mRFP-MeCP2 expression in BLA neurons was shown by immunohistochemistry of GFP (green) and mRFP (red), respectively. Induction of MeCP2 SUMOylation upon MeCP2 overexpression was confirmed in the BLA by in vitro SUMOylation assay. Results are depicted in FIG. 6.

The total number of chamber entries was similar among the five groups of animals (FIG. 6a), which indicated that the respective locomotor activity levels were similar. Result from social ability test revealed that animals in all the groups spent more time sniffing to stranger 1 than to the empty compartment and their sniffing time to stranger 1 was also similar (FIG. 6b). Social novelty test was conducted 10 min after the social ability test. Results indicated that control animals spent more time sniffing to stranger 2 than to stranger 1, whereas the Mecp2 cKO mice spent less time sniffing to stranger 2 compared with the control animals. But for Mecp2 cKO mice transducted with the lenti-mRFP-MeCP2WT vector, their sniffing time to stranger 2 was significantly increased compared to that of the Mecp2 cKO mice. However, if the Mecp2 cKO mice were transducted with the lenti-mRFP-MeCP2K412R vector, their sniffing time to stranger 2 was again decreased compared to that of Mecp2 cKO mice transducted with the lenti-mRFP-MeCP2WT vector and is comparable to that of Mecp2 cKO mice. When the Mecp2 cKO mice were transducted with the lenti-mRFP-MeCP2WT-SUMO1 fusion vector, their sniffing time to stranger 2 increased, as compared to that of Mecp2 cKO mice transducted with the lenti-mRFP-MeCP2K412R vector and was comparable to that of control animals (FIG. 6c).

Because cognitive impairment is another behavioral deficit observed in RTT patients and in mouse model of RTT, the issue of whether animals performed better in the social interaction task exhibited better memory retention was investigated. The same animals were subject to the cued fear conditioning learning task seven days after the social novelty test. Their memory retention capability was measured 24 hrs later. Results revealed that the Mecp2 cKO mice exhibited impaired fear memory, as compared with those of the control animals. However, if the Mecp2 cKO mice were transducted with the lenti-mRFP-MeCP2WT vector, their memory performance improved significantly, as compared with that of the Mecp2 cKO mice, and was comparable to that of control animals. By contrast, if the Mecp2 cKO mice were transducted with the lenti-mRFP-MeCP2K412R vector, their memory performance was worse than that of control animals, but was better than that of the Mecp2 cKO mice. Further, when the Mecp2 cKO mice were transducted with the lenti-mRFP-MeCP2WT-SUMO1 fusion vector, their memory performance was significantly improved, as compared with Mecp2 cKO mice transducted with the lenti-mRFP-MeCP2K412R vector, and was comparable to that of Mecp2 cKO mice transducted with the lenti-mRFP-MeCP2WT vector (FIG. 6d).

To demonstrate that Cre expression in the amygdala leads to successful depletion of MeCP2 expression, the BLA tissue of animals from the control group and Mecp2 cKO group were subject to western blot determination of MeCP2 expression. Results revealed that recombinase Cre transduction led to a significant decrease (approximately 75%) of MeCP2 expression in BLA neurons (FIG. 6e). The results indicated that the Mecp2 cKO mice transducted with the lenti-mRFP-MeCP2K412R vector had better memory performance, as compared with that of the Mecp2 cKO mice. One plausible explanation is that the overexpressed MeCP2K412R may still be phosphorylated, and thus leads to the signal transduction mediating fear memory formation. To test this hypothesis, animals from the Mecp2 cKO+vector group, Mecp2 cKO+MeCP2WT group and Mecp2 cKO+MeCP2K412R group were sacrificed after the fear memory test and their BLA tissues were subject to western blot determination for pS421MeCP2 and MeCP2. Results revealed that the expression level of MeCP2 increased for nearly 2.5 fold in both lenti-mRFP-MeCP2WT and lenti-mRFP-MeCP2K412R transduction groups. Yet, the phosphorylation level of MeCP2 still increased for about 16% in both groups (FIG. 6f). Since the same animals were subject to both social interaction and fear conditioning learning, we then examined whether there was a correlation between the performances of these two behaviors. Further analysis revealed that there was a significant correlation between individual scores for the time spent sniffing to stranger 2, and individual scores of fear memory performance (data not shown).

Since Lys-223 was suggested as a candidate SUMO site on MeCP2, the effect of MeCP2K223R on social interaction behavior in rats was investigated. Animals received Flag-MeCP2WT or Flag-MeCP2K223R transfection to their BLA area, and social interaction behaviors were measured 48 h later. Results revealed that MeCP2K223R transfection did not affect motor activity, social ability and social novelty performance in these animals (data not shown).

The above results showed that neuronal activation (NMDA injection) increased MeCP2 SUMOylation, and MeCP2 phosphorylation (at Ser-421 and Thr-308) facilitates MeCP2 SUMOylation (FIG. 3b), however, it is not known whether lack of SUMOylation would affect MeCP2-mediated neuronal plasticity. This issue was examined here by adopting both the high frequency stimulation (HFS) paradigm and theta-burst stimulation (TBS) paradigm of long-term potentiation (LTP) recording. The Mecp2 cKO mice were transducted with lenti-mRFP-vector, lenti-mRFP-MeCP2WT vector or lenti-mRFP-MeCP2K412R vector to their CA1 area. The Mecp2 loxp mice receiving mRFP-vector transduction served as the control. They were sacrificed 7 days later and their hippocampal tissue slices were subject to extracellular recording of field excitatory postsynaptic potential (fEPSP). Results revealed that when HFS paradigm was used, the induction and expression of LTP was significantly impaired in Mecp2 cKO mice, as compared with that of the Mecp2 loxp mice. Overexpression of MeCP2WT in Mecp2 cKO mice rescued this LTP impairment, but overexpression of MeCP2K412R failed to do so. However, the early induction of LTP (first 10 min) was less affected in Mecp2 cKO mice overexpressed with MeCP2K412R compared with that of the Mecp2 loxp mice (FIG. 6g). Similar results were obtained with the TBS paradigm (FIG. 6h).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for treating a subject having a neurodevelopmental disorder comprising administering to the subject an effective amount of a methyl-CpG-binding protein 2 (MeCP2) or a nucleic acid encoding the MeCP2 to alleviate or ameliorate the symptoms associated with the neurodevelopmental disorder.

2. The method of claim 1, wherein the MeCP2 has one or more post-translationa modifications that result in increased levels of sumoylation, phosphorylation or both, as compared with that of the endogenous MeCP2 in the subject.

3. The method of claim 2, wherein the post-translational modification corresponds in to sumoylation of the amino acid at position 412 in the wild-type MeCP2.

4. The method of claim 2, wherein the post-translational modification corresponds to phosphorylation of the amino acid at positions 308 or 421 in the wild-type MeCP2.

5. The method of claim 2, wherein the MeCP2 is administered to the subject in the amount of 0.001-100 mg/Kg.

6. The method of claim 2, wherein the nucleic acid is an expression vector.

7. The method of claim 6, wherein the expression vector is derived from a virus that is selected from the group consisting of, a herpes virus, a retrovirus, a vaccinia virus, an attenuated vaccinia virus, a canary pox virus, an adenovirus, and an adeno-associated virus.

8. The method of claim 1, wherein the neurodevelopmental disorder is any of attention deficit hyperactivity disorder (ADHD), schizophrenia, obsessive-compulsive disorder (OCD), mental retardation, autistic spectrum disorders, cerebral palsy, articulation disorder, Rett syndrome, or learning disabilities.

9. The method of claim 8, wherein the neurodevelopmental disorder is Rett syndrome.

10. The method of claim 9, further comprising administering an effective amount of N-methyl-D-aspartate (NMDA), an insulin-like growth factor (IGF-1) or corticotropin-releasing factor (CRF) to the subject.

11. The method of claim 1, wherein the subject is human.


SEQUENCE LISTING (19 sequences)

SEQ ID NO: 1 - DNA, 35 bp, Artificial Sequence
  Mecp2 cDNA primer-1
atttgcggccgccaccatggtagctgggatgttag

SEQ ID NO: 2 - DNA, 27 bp, Artificial Sequence
  Mecp2 cDNA primer-2
taaccgcgggctaactctctcggtcac

SEQ ID NO: 3 - DNA, 34 bp, Artificial Sequence
  construct_forward primer
atcgggatccgccaccatggcctcctccgaggac

SEQ ID NO: 4 - DNA, 31 bp, Artificial Sequence
  mRFP_reverse primer
atcgcctaggttaggcgccggtggagtggcg

SEQ ID NO: 5 - DNA, 34 bp, Artificial Sequence
  mRFP-Mecp2WT, mRFP-Mecp2K412R_reverse primer
atcgcctaggtcagctaactctctcggtcacggg

SEQ ID NO: 6 - DNA, 33 bp, Artificial Sequence
  mRFP-Mecp2-SUMO1, mRFP-Mecp2WT-SUMO1_reverse primer
atcgcctaggctaaaccgtcgagtgaccccccg

SEQ ID NO: 7 - DNA, 49 bp, Artificial Sequence
  Cre vector_forward primer
atcggaattcccaaagaagaagagaaaggttatgtccaatttactgacc

SEQ ID NO: 8 - DNA, 30 bp, Artificial Sequence
  Cre vector_reverse primer
atcggcggccgcctaatcgccatcttccag

SEQ ID NO: 9 - DNA, 37 bp, Artificial Sequence
  GFP vector_forward primer
atcgagtactgccaccatggagatcgagtgccgcatc

SEQ ID NO: 10 - DNA, 29 bp, Artificial Sequence
  GFP vector_reverse primer
atcgggtaccggcgaaggcgatgggggtc

SEQ ID NO: 11 - DNA, 51 bp, Artificial Sequence
  DNA oligonucleotide
caatgccctggaacggaattcttctaataaaagatgtatcattttaaatgc

SEQ ID NO: 12 - DNA, 51 bp, Artificial Sequence
  DNA oligonucleotide_sense strand
agagattgcctgacgtcagagagctaggattgcctgacgtcagagagctag

SEQ ID NO: 13 - DNA, 51 bp, Artificial Sequence
  DNAoligonucleotide_antisense strand
ctagctctctgacgtcaggcaatcctagctctctgacgtcaggcaatctct

SEQ ID NO: 14 - DNA, 20 bp, Artificial Sequence
  GAPDH_forward primer
ggcaagttcaatggcacagt

SEQ ID NO: 15 - DNA, 22 bp, Artificial Sequence
  GAPDH_reverse primer
tggtgaagacgccagtagactc

SEQ ID NO: 16 - DNA, 19 bp, Artificial Sequence
  Bdnf_forward primer
ctaggactggaagtggaaa

SEQ ID NO: 17 - DNA, 19 bp, Artificial Sequence
  Bdnf_reverse primer
atttcatgctagctcgccg

SEQ ID NO: 18 - DNA, 21 bp, Artificial Sequence
  combined DNA/RNA molecule
  misc_RNA 20,21: /note=“t is DNA nucleotide-thymine”
uccggaucauucuagagcutt

SEQ ID NO: 19 - DNA, 21 bp, Artificial Sequence
  combined DNA/RNA molecule
  mRNA 20,21: /note=“t is DNA nucleotide-thymine”
agcucuagaaugauccggatt